SEQUENCE LISTING (36 sequences)

SEQ ID NO: 1 - RNA, 89 nt, Homo sapiens
gucagcagugccuuagcagcacguaaauauuggcguuaagauucuaaaauuaucuccaguauuaacugugcugcugaaguaagguugac

SEQ ID NO: 2 - RNA, 22 nt, Homo sapiens
uagcagcacguaaauauuggcg

SEQ ID NO: 3 - RNA, 22 nt, Homo sapiens
ccaguauuaacugugcugcuga

SEQ ID NO: 4 - RNA, 98 nt, Homo sapiens
uugaggccuuaaaguacuguagcagcacaucaugguuuacaugcuacagucaagaugcgaaucauuauuugcugcucuagaaauuuaaggaaauucau

SEQ ID NO: 5 - RNA, 22 nt, Homo sapiens
uagcagcacaucaugguuuaca

SEQ ID NO: 6 - RNA, 22 nt, Homo sapiens
cgaaucauuauuugcugcucua

SEQ ID NO: 7 - RNA, 87 nt, Homo sapiens
agcuucccuggcucuagcagcacagaaauauuggcacagggaagcgagucugccaauauuggcugugcugcuccaggcaggguggug

SEQ ID NO: 8 - RNA, 21 nt, Homo sapiens
uagcagcacagaaauauuggc

SEQ ID NO: 9 - RNA, 22 nt, Homo sapiens
ccaauauuggcugugcugcucc

SEQ ID NO: 10 - RNA, 81 nt, Homo sapiens
guucugugaggggcucacaucaccccaucaaaguggggacucauggggagaggggguaguuaggagcuuugauagaggcgg

SEQ ID NO: 11 - RNA, 18 nt, Homo sapiens
gggcucacaucaccccau

SEQ ID NO: 12 - RNA, 92 nt, Homo sapiens
cggcuggacagcgggcaacggaaucccaaaagcagcuguugucuccagagcauuccagcugcgcuuggauuucguccccugcucuccugccu

SEQ ID NO: 13 - RNA, 23 nt, Homo sapiens
caacggaaucccaaaagcagcug

SEQ ID NO: 14 - RNA, 22 nt, Homo sapiens
gcugcgcuuggauuucgucccc

SEQ ID NO: 15 - DNA, 70 bp, Homo sapiens
tgttcttgtagttgaaatacaatgatggtttttcatatcattggtcgtggttgtagcccgtgcaagaata

SEQ ID NO: 16 - RNA, 23 nt, Homo sapiens
ugguuguaguccgugcgagaaua

SEQ ID NO: 17 - RNA, 65 nt, Homo sapiens
cuugaauccuuggaaccuaggugugagugcuauuucagugcaacacaccuauucaaggauucaaa

SEQ ID NO: 18 - RNA, 24 nt, Homo sapiens
aauccuuggaaccuaggugugagu

SEQ ID NO: 19 - RNA, 22 nt, Homo sapiens
aacacaccuauucaaggauuca

SEQ ID NO: 20 - RNA, 71 nt, Homo sapiens
gaugucccuguuugucccuguucaggcgccaccuguggcugucugccacaaguacuauuugagaccaucac

SEQ ID NO: 21 - RNA, 18 nt, Homo sapiens
gucccuguucaggcgcca

SEQ ID NO: 22 - RNA, 98 nt, Homo sapiens
uugggcaaggugcggggcuagggcuaacagcagucuuacugaagguuuccuggaaaccacgcacaugcuguugccacuaaccucaaccuuacucgguc

SEQ ID NO: 23 - RNA, 22 nt, Homo sapiens
ugcggggcuagggcuaacagca

SEQ ID NO: 24 - RNA, 22 nt, Homo sapiens
cuguugccacuaaccucaaccu

SEQ ID NO: 25 - RNA, 22 nt, Homo sapiens
uagcuuaucagacugauguuga

SEQ ID NO: 26 - RNA, 21 nt, Homo sapiens
caacaccagucgaugggcugu

SEQ ID NO: 27 - RNA, 76 nt, Homo sapiens
ucuuuacucccacugcuucacuugacuagccuuuaaaaaagaaaggcuugguuugaugaaugggugagagaaaagg

SEQ ID NO: 28 - RNA, 22 nt, Homo sapiens
cucccacugcuucacuugacua

SEQ ID NO: 29 - RNA, 75 nt, Homo sapiens
aguugguccgaguguuguggguuauuguuaaguugauuuaacauugucuccccccacaaccgcgcuugacuagcu

SEQ ID NO: 30 - RNA, 25 nt, Homo sapiens
cccccacaaccgcgcuugacuagcu

SEQ ID NO: 31 - RNA, 80 nt, Homo sapiens
ugagaggccgcaccuugccuugcugcccgggccgugcacccgugggccccagggcgacgcggcgggggcggcccuagcga

SEQ ID NO: 32 - RNA, 20 nt, Homo sapiens
ccccagggcgacgcggcggg

SEQ ID NO: 33 - RNA, 20 nt, Homo sapiens
ccccagggcgacgcggcggg

SEQ ID NO: 34 - RNA, 95 nt, Homo sapiens
auagcuguugugucacuuccucaugcugacauauuuacuagaggguaaaauuaauaaccuucuaguaagaguggcagucgaagggaagggcucau

SEQ ID NO: 35 - RNA, 21 nt, Homo sapiens
ucuaguaagaguggcagucga

SEQ ID NO: 36 - RNA, 72 nt, Homo sapiens
ugucggguagcuuaucagacugauguugacuguugaaucucauggcaacaccagucgaugggcugucugaca

(19) United States
(12) Patent Application Publication
(10) Pub. No.: US 2015/0080229 A1
(43) Pub. Date: Mar. 19, 2015

(54) PLASMA MICRORIBONUCLEIC ACIDS AS BIOMARKERS FOR ENDOMETRIOSIS AND ENDOMETRIOSIS-ASSOCIATED OVARIAN CANCER

(71) Applicant: University of Pittsburgh - of the Commonwealth System of Higher Education, Pittsburgh, PA (US)

(72) Inventors: Xin Huang, Wexford, PA (US); Swati Suryawanshi, Pittsburgh, PA (US); Robert Page Edwards, Gibsonia, PA (US); Anda Mioara Vlad, Pittsburgh, PA (US)

(73) Assignee: University of Pittsburgh - of the Commonwealth System of Higher Education, Pittsburgh, PA (US)

(21) Appl. No.: 14/495,561

(22) Filed: Sep. 24, 2014

Related U.S. Application Data
(63) Continuation of application No. PCT/US13/30382, filed on Mar. 12, 2013.
(60) Provisional application No. 61/617,532, filed on Mar. 29, 2012.

(51) Int. Cl. C12Q 1/68

(52) U.S. Cl. CPC C12Q 1/6886; C12Q 2600/178; C12Q 2600/16
USPC 506/2; 435/6.12; 506/16; 506/9

(57) ABSTRACT

The present invention relates to methods and compositions for differentiating between absence of disease, endometriosis, and EAOC or serous ovarian cancer in a subject. It is based, at least in part, on the discovery that certain microRNAs are associated with each of these conditions.

### CROSS REFERENCE TO RELATED APPLICATIONS

The present application is a continuation of PCT/U.S. Ser. No. 13/030,382, filed Mar. 12, 2013, and claims priority to U.S. Provisional Application No. 61/617,532, filed on Mar. 29, 2012, the disclosures of both of which are incorporated by reference herein in their entireties.

### GRANT INFORMATION

This invention was made with government support under Grant No. 1 ROI CA163462-01 awarded by the National Institutes of Health. The government has certain rights in the invention.

### 1. INTRODUCTION

The present invention relates to plasma miRNAs for use as biomarkers for—and to distinguish between—endometriosis, endometriosis-associated ovarian cancer (EAOC), and serous ovarian carcinoma.

### 2. BACKGROUND OF THE INVENTION

Epithelial ovarian cancer (EOC) is often referred to as the ‘silent killer’ since its early stages are difficult to detect and the majority of patients are usually diagnosed with advanced disease. Although the 5 year survival rate of women with early stage ovarian cancer is 90%, it plunges down to 11% in patients with late stage cancer [51]. The key to increasing the overall survival rate of women with ovarian cancer lies in early detection and screening, especially of patients with precursor lesions.

Endometriosis, a chronic inflammatory disease, is the most speculated precursor of ovarian epithelial cancers, especially those with endometrioid and clear cell histology [15, 16, 42, 52-57]. Defined as a common gynecological disorder affecting up to 10% to 15% of women in the reproductive age group, endometriosis consists of endometrial-like ectopic epithelial glands surrounded by stroma, found at locations outside uterine cavity. While largely benign, the lesions often show characteristics similar to those of malignancy such as cell proliferation, invasion, tissue remodeling and neovascularization. Despite still being largely underappreciated as a cancer precursor, progressively accumulating evidence from epidemiological and molecular studies demonstrates the role of endometriosis as a potential precursor of endometrioid and clear cell ovarian cancers, also known as endometriosis-associated ovarian cancer (EAOC) [58, 59].

For early detection of EOC, two major screening methods have been tested: transvaginal sonography (TVS) and serum protein biomarker testing [2], although both approaches have been found to have low specificity and sensitivity for early EOC screening and thus not recommended for screening the general population with average risk [2]. CA-125 is the most widely used serum biomarker in EOC [4]. However, less than 50% of early stage EOC patients have elevated CA-125 levels, and elevated circulating CA-125 levels can result from many other medical/physiological conditions [2]. Thus, CA-125 has been mostly used for monitoring EOC progression [5].

As with early stage ovarian cancer, diagnosis of endometriosis is often difficult and can involve invasive procedures (laparoscopy or laparotomy). Even in diagnosed patients, it remains a challenge to predict who is at risk to develop EAOC later in life, demonstrating the urgent need to develop specific and non-invasive (preferably blood based) biomarker assays for early stage EOC and precursor endometriosis.

MicroRNAs (miRNAs) are single-stranded small RNA molecules that regulate gene expression by inhibiting mRNA translation or by facilitating cleavage of the target mRNA [6]. Recently, microRNA (miRNA) expression profiles have been utilized to accurately classify normal and cancerous tissues as well as subtypes of malignancies with superiority to messenger RNA (mRNA) expression profiling [10]. Certain miRNAs have been reported to be associated with ovarian cancer [76].

Although tissue miRNA expression signatures have shown great promise as a new class of biomarkers, the fact that they are based on tissue samples weighs against their use in early diagnosis of endometriosis and/or EAOC. In contrast, blood based miRNA expression profiling has several unique advantages, such as easy (relatively non-invasive) access, superior stability of miRNAs in blood, and the potential for developing a screening test for a large population.

### 3. SUMMARY OF THE INVENTION

The present invention relates to methods and compositions for differentiating between absence of disease, endometriosis, and EAOC or serous ovarian cancer in a subject. It is based, at least in part, on the discovery that certain microRNAs are associated with each of these conditions.

### 4. BRIEF DESCRIPTION OF THE FIGURES

FIG. 1. Experimental design.

FIG. 2A-C. Reliability of RNA extraction and reproducibility of RT-qPCR techniques. A) Reliability of our RNA extraction technique was tested by adding various concentrations of synthesized miR-210 RNA to plasma samples, followed by RNA extraction, RT, and qPCR. Left panel, serial dilution of synthesized miR-210 spiked into patient plasma prior to RNA extraction could be detected linearly. Since the naked synthesized miRNA is prone to degradation by RNases in plasma, synthesized miR-210 was mixed with LipoFectamine before spiked in plasma, which protected synthesized miR-210 from degradation; Right panel, dissociation curve analysis demonstrates the specificity of our PCR amplification. B) Reproducibility of our techniques, including RNA extraction, RT, and qPCR, was confirmed by performing three independent runs of three miRNAs in six plasma samples. Similar CT values with only small variations are seen across each of the three runs. These results indicate that miRNAs can be efficiently extracted and amplified from plasma and RT-qPCR results of plasma miRNAs can be reliably compared across multiple samples. C) Reproducibility of our RT-qPCR assay using three consecutive plasma samples collected one month apart from each of three preoperative EAOC patients. These results indicate that plasma miRNAs can be reliably measured in different plasma samples from the same patient.

FIG. 3A-B. Reliability of RT-qPCR assays. A) Expression of miR-16, 21, and 195 in three plasma samples collected in heparinized tubes and in matched serum samples collected in tubes without anticoagulant from EAOC patients. B) No difference is detected for plasma miR-132 expression (CT) between healthy control samples purchased from Innovative Research Labs and plasma samples from endometriosis and EAOC patients collected at Magee-Womens Hospital by RT-qPCR. Average CT from each group is shown. Error bar, standard deviation.

FIG. 4. miR-132 demonstrates consistent CT values (ranging from 27 to 29.5.) across all categories of plasma samples (healthy controls, n=20; endometriosis, n=33; EAOC, n=14; and SOC, n=21), and was used for normalization of RT-qPCR results in this study.

FIG. 5A-B. Plasma miRNA expression profiles can distinguish different disease categories. A) Unsupervised hierarchical clustering was applied to miRNAs with <30% missing values in healthy controls, endometriosis, and EAOC samples (n=20, 33, and 14, respectively). Different distance measure and link were explored. Samples are classified into three clusters based on the expression signature of 23 plasma miRNAs. B) Principal component analysis was applied to markers with adjusted p value <0.2 in either one of the three groups' pair-wised comparisons. First three components were used for the three-dimensional plot. Norm, healthy controls; Endo, endometriosis.

FIG. 6A-F. Unsupervised hierarchical clustering analysis of samples based on the 23-miRNA expression profiles. A) Clustering of all four groups (healthy controls, n=20; endometriosis, n=33; EAOC, n=14; and SOC, n=21). Pair-wised clustering of B) endometriosis and EAOC, C) endometriosis and SOC, D) EAOC and SOC, E) healthy controls and EAOC, and F) healthy controls and SOC.

FIG. 7A-F. Box plots of top three differentially expressed miRNAs in pair-wised comparisons. y-axis, log₂ of folder changes of plasma miRNA expression (log₂(FC)). Norm, healthy controls; Serous, SOC; Endo, endometriosis. A) healthy versus endometriosis; B) healthy versus EAOC; C) endometriosis versus EAOC; D) endometriosis versus serous ovarian carcinoma; E) endometriosis versus serous ovarian carcinoma; F) EAOC versus serous ovarian carcinoma.

FIG. 8A-F. The leave-one-out cross validation receiver operating characteristic (ROC) curves of Logistic regression model for four groups' pair-wised comparisons are plotted based on the top three markers. Area under curve (AUC) is also provided. SN, sensitivity; SP, specificity. A) healthy versus endometriosis; B) healthy versus EAOC; C) endometriosis versus EAOC; D) EAOC versus serous ovarian carcinoma; E) healthy versus serous ovarian carcinoma; and F) endometriosis versus SOC.

FIG. 9. Increased expression of plasma miRNAs along with the progression of diseases from endometriosis to EAOC, but not in SOC samples. The eight miRNAs are derived from shared top 10 most differentially expressed miRNAs in both endometriosis and EAOC samples compared to healthy controls. y-axis, log₂ of folder changes of plasma miRNA expression (log₂(FC)). Norm, healthy controls; Serous, SOC; Endo, endometriosis.

FIG. 10A-B. The plasma miRNA expression signature that differentiates healthy controls from EAOC patients can be detected in a mouse endometrioid ovarian cancer model. A) Left panel shows ovarian tumor at the Ad-Cre injected site (arrow), but no tumor formation seen on non-injected left ovary. Middle panel is 10×H&E staining of a cross section of mouse ovarian tumor with ovary (Ov), oviduct (Od), and ovarian tumor (OvT). Right panel shows magnified image of ovarian tumor with endometrioid histology (40×). B) Expression of orthologous miRNAs in healthy mice (n=5) and mice with EAOC (n=6). Four out of the five miRNAs are significantly upregulated in most of the mice with EAOC as compared to normal (p=0.00009, 0.000433, 0.000209, and 0.00014 for miR-16, 21, 15b, and 195, respectively; student's t-test), similar to the profiles in human EAOC samples. miR-191 did not reach statistical significance, although there is also a trend that expression of plasma miR-191 is elevated in EAOC mice compared to that of in normal mice. Equal amount of RNA extracted from mouse serum was used for RT and qPCR. The raw CT values were plotted because currently there is no consensus on endogenous plasma miRNA in mouse that can be used for normalization. C, mice with EAOC tumors; N, normal control mice.

FIG. 11. NanoString analysis reveals very low correlation of miRNA expression between matching tissue and plasma of EAOC and endometriosis patients. Left panel, comparison of three matched endometriosis tissue and plasma samples; Right panel, comparison of three matched EAOC tissue and plasma samples. The NanoString data were normalized using the nSolver software. x-axis, copy number of miRNAs in tissue samples; y-axis, copy number of miRNAs in plasma samples. Endo, endometriosis.

FIG. 12. Lack of correlation for miRNA expression between matching tumor tissue and plasma samples from five tumor-bearing LSL-Kras^{G12D/+}/Pten^{loxp/loxp} mice. Expression of 10 mouse miRNAs (mmu-miR-15b, 16, 21, 132, 191, 195, 362-5p, 652, 744, and 1274a) were examined by RT-qPCR.

FIG. 13A-B. (A) Scatter plot of CA-125 after ELISA analysis on 86 plasma samples shows upregulation of CA-125 in most serous ovarian cancer samples. (B) CA-125 levels can differentiate serous from normal, endometriosis, and EAOC cases with significant p-values. However, CA-125 also suffers high non-specificity as demonstrated in this FIG.

### 5. DETAILED DESCRIPTION OF THE INVENTION

For clarity, the detailed description is divided into the following subsections:

plasma biomarkers that distinguish between a healthy subject and a subject with endometriosis;

(ii) plasma biomarkers that distinguish between a subject with endometriosis and a subject with endometriosis-associated ovarian cancer;

(iii) plasma biomarkers that distinguish between a subject with serous ovarian cancer and a subject with endometriosis-associated ovarian cancer;

(iv) plasma biomarkers that distinguish between a healthy subject and a subject with serous ovarian cancer;

(v) plasma biomarkers that distinguish between a healthy subject and a subject with EAOC;

(vi) plasma biomarkers that distinguish between an endometriosis subject and a subject with serous ovarian cancer; and

(vii) correlative kits and methods.

A subject is a human female subject. The miRNAs discussed herein are human miRNAs, the sequences of which are known and publicly available. Subjects who may benefit from this invention include, but are not limited to, subjects with pelvic pain, infertility, menorrhagia, metromenorrhagia, a family history of ovarian or breast cancer, a suspicious Pap smear, or BRCA2 positive status.

This disclosure relates to plasma mir biomarkers, meaning microribonucleic acids occurring in the plasma. Preferably measurements are performed on plasma samples although serum also may be used. Measuring the level of miRNA may comprise purifying nucleic acid from the plasma/serum sample.

Microribonucleic acid (“miRNA” or, when used to name a specific microriobnucleic acid, “mir” or “miR”) may be measured by any method known in the art. As non-limiting examples, assays for measuring miRNA are described in Ach et al., BMC Biotechnology 2008, 8:69 (77), including quantitative RT-PCR (qPCR) analysis (78-80); high-throughput sequencing of small RNA libraries (81), microarray analysis (82-87) and analysis by Nanostring Technologies.

Non-limiting examples of means for measuring (or “measurement means” for) miRNA, as discussed in the sections below, include, for qPCR, (i) stem-loop reverse transcriptase (“RT”) primers which may be used together with TaqMan PCR (Applied Biosystems) analysis (78-80); (ii) locked nucleic acid primers (Exiqon; 80); and/or (iii) materials for poly(A) tailing (QIAGEN, Stratagene). Means for measuring miRNA by microarray analysis may comprise, for example, an array of complementary nucleic acids bound to a solid substrate (for example, see 82-87).

Primers for use in qPCR may be designed based on the sequences for said miRNAs, which are known in the art. A primer may comprise DNA nucleotides, and may be, for example and without limitation, between about 5 and about 30 or preferably between about 10 and 25 or between about 15 and 20 nucleotides in length, and may be capable of amplifying the target miRNA; the primer, for example, and without limitation, may comprise a region at least 10 or at least 12 or at least 13 or at least 14 or at least 15 nucleotides in length which is complementary to a portion of the miRNA itself and/or complementary to nucleic acid sequence flanking the miRNA sequence to be amplified.

The sequences of miRNAs referred to herein are known in the art and publicly available. Non-limiting examples of nucleotide sequences of a subset of miRNAs referred to herein are provided in Table 1, below. Oligonucleotide primers as discussed above may be prepared based on these sequences for use in qPCR to amplify said miRNAs, which can be used to measure plasma levels. In specific non-limiting embodiments, the primers may amplify mature miRNA sequence.

A group of biomarkers recited as “mir A alone or with at least one or more of mir B and mirC” means that the biomarkers used may include (i) (only) mir A; (ii) mir A and mir B; (iii) mir A and mir C or (iv) mir A, mir B and mir C.

In non-limiting embodiments of the invention, levels of biomarkers may be evaluated using a microarray, for example attached to a solid support such as a chip or a bead or population of beads or analogous structures.

TABLE 1
mir 16
stem loop
GUCAGCAGUGCCUUAGCAGCACGUAAAUAUUGGCGUUAA
GAUUCUAAAAUUAUCUCCAGUAUUAACUGUGCUGCUGAAGUAAGGUUGAC
(SEQ ID NO: 1)
mature :
Acc. No.: MIMAT0000069 UAGCAGCACGUAAAUAUUGGCG
(SEQ ID NO: 2)
Ace. No.: MIMAT0004489 CCAGUAUUAACUGUGCUGCUGA
(SEQ ID NO: 3)
mir 15b
stem-loop UUGAGGCCUUAAAGUACUGUAGCAGCACAUCAUGGUUUAC
AUGCUACAGUCAAGAUGCGAAUCAUUAUUUGCUGCUCUAGAAAUUUAAGG
AAAUUCAU (SEQ ID NO: 4)
mature:
Ace. No.: MIMAT0000417 UAGCAGCACAUCAUGGUUUACA
(SEQ ID NO: 5)
Ace. No.: MIMAT0004586 CGAAUCAUUAUUUGCUGCUCUA
(SEQ ID NO: 6)
mir 195
stem-loop: AGCUUCCCUGGCUCUAGCAGCACAGAAAUAUUGGCA
CAGGGAAGCGAGUCUGCCAAUAUUGGCUGUGCUGCUCCAGGCAGGGUGGU
G (SEQ ID NO: 7)
mature:
Ace. No.: MIMAT0000461 UAGCAGCACAGAAAUAUUGGC
(SEQ ID NO: 8)
Acc. No. MIMAT0004615 CCAAUAUUGGCUGUGCUGCUCC
(SEQ ID NO: 9)
mir 4284
stem-loop:
GUUCUGUGAGGGGCUCACAUCACCCCAUCAAAGUGGGGACUCAUGGGGAG
AGGGGGUAGUUAGGAGCUUUGAUAGAGGCGG (SEQ ID NO: 10)
mature:
Acc. No.: MIMAT0016915 GGGCUCACAUCACCCCAU
(SEQ ID NO: 11)
mir 191
stem-loop:
CGGCUGGACAGCGGGCAACGGAAUCCCAAAAGCAGCU
GUUGUCUCCAGAGCAUUCCAGCUGCGCUUGGAUUUCGUCCCCUGCUCUCC
UGCCU
(SEQ ID NO: 12)
mature:
Ace. No.: MIMAT0000440 CAACGGAAUCCCAAAAGCAGCUG
(SEQ ID NO: 13)
Ace. No. MIMAT0001618 GCUGCGCUUGGAUUUCGUCCCC
(SEQ ID NO: 14)
mir 1974
stem-loop:
tgttcttgtagttgaaatacaatgatggtttttcatatcattggtcgtgg
ttgtagcccgtgcaagaata (SEQ ID NO: 15)
mature:
Acc. No: MI0009984 UGGUUGUAGUCCGUGCGAGAAUA
(SEQ ID NO:16)
mir 362-5p
stem-loop:
CUUGAAUCCUUGGAACCUAGGUGUGAGUGCUAUUUCAGUGCAA
CACACCUAUUCAAGGAUUCAAA (SEQ ID NO: 17)
mature:
Acc No: MIMAT0000705: AAUCCUUGGAACCUAGGUGUGAGU
(SEQ ID NO: 18)
Acc. No: MIMAT0004683 AACACACCUAUUCAAGGAUUCA
(SEQ ID NO: 19)
mir 1274a
stem-loop:
GAUGUCCCUGUUUGUCCCUGUUCAGGCGCCACCUGUGGCUGUCUGCCACA
AGUACUAUUUGAGACCAUCAC (SEQ ID NO: 20)
mature:
Ace. No: MI0006410 GUCCCUGUUCAGGCGCCA
(SEQ ID NO: 21)
mir 744
stem-loop:
UUGGGCAAGGUGCGGGGCUAGGGCUAACAGCAGUCUUACUGAA
GGUUUCCUGGAAACCACGCACAUGCUGUUGCCACUAACCUCAACCUUACU
CGGUC
(SEQ ID NO: 22)
mature:
Acc. No. MIMAT0004945 UGCGGGGCUAGGGCUAACAGCA
(SEQ ID NO: 23)
Acc. No. MIMAT0004946 CUGUUGCCACUAACCUCAACCU
(SEQ ID NO: 24)
mir 21
stem-loop:
UGUCGGGUAGCUUAUCAGACUGAUGUUGACUGUUGAAUCUCA
UGGCAACACCAGUCGAUGGGCUGUCUGACA (SEQ ID NO: 36)
mature:
Ace. No. MIMAT0000076 UAGCUUAUCAGACUGAUGUUGA
(SEQ ID NO: 25)
Acc. No. MIMAT0004494 CAACACCAGUCGAUGGGCUGU
(SEQ ID NO: 26)
mir 1979
stem-loop:
UCUUUACUCCCACUGCUUCACUUGACUAGCCUUUAAAAAAGAAAGGCUUG
GUUUGAUGAAUGGGUGAGAGAAAAGG (SEQ ID NO: 27)
mature:
Acc. No.: M10009989 CUCCCACUGCUUCACUUGACUA (SEQ ID
NO: 28)
mir 1975
stem-loop:
AGUUGGUCCGAGUGUUGUGGGUUAUUGUUAAGUUGAUUUAACAUUGUCUC
CCCCCACAACCGCGCUUGACUAGCU
(SEQ ID NO: 29)
mature:
Acc. No.: MI0009985 CCCCCACAACCGCGCUUGACUAGCU
(SEQ ID NO: 30)
mir 1915
stem-loop:
UGAGAGGCCGCACCUUGCCUUGCUGCCCGGGCCGUGCACCCGUGGGCCCCA
GGGCGACGCGGCGGGGGCGGCCCUAGCGA (SEQ ID NO: 31)
mature:
Acc. No.: MIMAT0007892 ccccagggc gacgcggcggg
(SEQ ID NO: 32)
Acc. No.: MIMAT0007891 ccccagggcgacgcggcggg
(SEQ ID NO: 33)
mir 628-3p
stem-loop:
AUAGCUGUUGUGUCACUUCCUCAUGCUGACAUAUUUACUAGAGGGUAAAA
UUAAUAACCUUCUAGUAAGAGUGGCAGUCGAAGGGAAGGGCUCAU
(SEQ ID NO: 34)
mature:
Acc. No.: MIMAT0003297 ucuaguaagaguggcagucga
(SEQ ID NO: 35)

### 5.1 Plasma Biomarkers that Distinguish Between a Healthy Subject and a Subject with Endometriosis

The present invention provides for the following plasma mir biomarkers which differ between a healthy subject and a subject with endometriosis.

The present invention relates to a method of diagnosing endometriosis in a subject, comprising measuring or having measured (i.e., directing measurement of) the plasma level of:

one or more or two or more or three or more or four or more of the following biomarkers: mir 16; mir 21; mir 15b; mir 191; mir 195; mir 652, mir 1308; mir 1915, mir 1973; mir 1974; mir 1977, mir 1978, mir 1979, mir 4284, mir 4313, and mir 362-5p; or

one or more or two or more or three or more or four or more of the following biomarkers: mir 16; mir 15b; mir 191; mir 195; mir 1973; mir 1974; mir 1977, mir 1978, mir 1979, mir 4284 and mir 362-5p; or

mir 15b and one or more of the following biomarkers: mir 16; mir 21; mir 191; mir 195; mir 652, mir 1308; mir 1915, mir 1973; mir 1974; mir 1977, mir 1978, mir 1979, mir 4284, mir 4313, and mir 362-5p; or

mir 16 and one or more of the following biomarkers: mir 21; mir 15b; mir 191; mir 195; mir 652, mir 1308; mir 1915, mir 1973; mir 1974; mir 1977, mir 1978, mir 1979, mir 4284, mir 4313, and mir 362-5p; or

mir 191 and one or more of the following biomarkers: mir 16; mir 21; mir 15b; mir 195; mir 652, mir 1308; mir 1915, mir 1973; mir 1974; mir 1977, mir 1978, mir 1979, mir 4284, mir 4313, and mir 362-5p; or

mir 195 and one or more of the following biomarkers: mir 16; mir 21; mir 15b; mir 191; mir 652, mir 1308; mir 1915, mir 1973; mir 1974; mir 1977, mir 1978, mir 1979, mir 4284, mir 4313, and mir 362-5p; or

mir 1974 and one or more of the following biomarkers: mir 16; mir 21; mir 15b; mir 191; mir 195; mir 652, mir 1308; mir 1915, mir 1973; mir 1977, mir 1978, mir 1979, mir 4284, mir 4313, and mir 362-5p; or

mir 4284 and one or more of the following biomarkers: mir 16; mir 21; mir 15b; mir 191; mir 195; mir 652, mir 1308; mir 1915, mir 1973; mir 1974; mir 1977, mir 1978, mir 1979, mir 4284, mir 4313, and mir 362-5p; or

mir 15b and one or more of, or at least two of, mir 16, mir 191, mir 195, mir 1974, and mir 4284; or

mir 16 and one or more of, or at least two of, mir 15b, mir 191, mir 195, mir 1974, and mir 4284; or

mir 191 and one or more of, or at least two of, mir 15b, mir 16, mir 195, mir 1974, and mir 4284; or

mir 195 and one or more of, or at least two of mir 15b, mir 16, mir 191, mir 1974, and mir 4284; or

mir 1974 and one or more of, or at least two of, mir 15b, mir 16, mir 191, mir 195, and mir 4284; or

mir 4284 and one or more of, or at least two of, mir 15b, mir 16, mir 191, mir 195, and mir 1974; or

mir 16, alone or with one or both of mir 195 and mir 191; or

mir 195, alone or with one or both of mir 16 and mir 191; or

mir 191, alone or with one or both of mir 16 and mir 195; or

mir 16, mir 195, and mir 191, optionally with at least one or more of mir 1974, mir 4284, mir-15b, mir-1978, mir-1979, mir-362-5p and mir 1973; or

mir 16, mir 195, and mir 191, optionally with at least one or more of mir 1974, mir 4284 and mir-15b;

and comparing the level(s) with the level(s) of said biomarker(s) in a healthy control (either by measuring the level in a plasma or blood sample from one or more healthy individuals or by comparing to a predetermined reference value obtained using one or more healthy individuals), where an increase in the level(s) of the biomarker(s) in the subject relative to the level(s) in the control indicates that the subject has endometriosis. In specific non-limiting embodiments, measurement of the level of miRNA may be performed by qPCR, Nanostring, microarray analysis, or next generation sequencing of RNA (eg miRNA) prepared from a plasma sample collected from the subject. In certain non-limiting embodiments, the diagnostic method may further comprise recommending or performing laparoscopic obtention of a tissue biopsy and/or recommending or performing a pelvic ultrasound or other imaging study (e.g. computer assisted tomography (CAT) scan, magnetic resonance imaging (MRI) or Positron Emmision Tomography (PET scan)) to further support the diagnosis. In certain non-limiting embodiments laparoscopy and/or imaging study may be performed if the plasma levels of the biomarker(s) is/are indicative of endometriosis.

In certain non-limiting embodiments, relative to healthy control, the plasma level in a subject with endometriosis is increased for: mir 16; mir 21; mir 15b; mir 191; mir 195; mir 652, mir 1308; mir 1915, mir 1973; mir 1974; mir 1977, mir 1978, mir 1979, mir 4284, mir 4313, and/or mir 362-5p.

The present invention provides for a method of treating a subject suffering from pelvic pain, menorrhagia, menometrorrhagia, and/or infertility, comprising measuring or having measured the plasma level of:

one or more or two or more or three or more or four or more of the following biomarkers: mir 16; mir 21; mir 15b; mir 191; mir 195; mir 652, mir 1308; mir 1915, mir 1973; mir 1974; mir 1977, mir 1978, mir 1979, mir 4284, mir 4313, and mir 362-5p; or

one or more or two or more or three or more or four or more of the following biomarkers: mir 16; mir 15b; mir 191; mir 195; mir 1973; mir 1974; mir 1977, mir 1978, mir 1979, mir 4284 and mir 362-5p; or

mir 15b and one or more of the following biomarkers: mir 16; mir 21; mir 191; mir 195; mir 652, mir 1308; mir 1915, mir 1973; mir 1974; mir 1977, mir 1978, mir 1979, mir 4284, mir 4313, and mir 362-5p; or

mir 16 and one or more of the following biomarkers: mir 21; mir 15b; mir 191; mir 195; mir 652, mir 1308; mir 1915, mir 1973; mir 1974; mir 1977, mir 1978, mir 1979, mir 4284, mir 4313, and mir 362-5p; or

mir 191 and one or more of the following biomarkers: mir 16; mir 21; mir 15b; mir 195; mir 652, mir 1308; mir 1915, mir 1973; mir 1974; mir 1977, mir 1978, mir 1979, mir 4284, mir 4313, and mir 362-5p; or

mir 195 and one or more of the following biomarkers: mir 16; mir 21; mir 15b; mir 191; mir 652, mir 1308; mir 1915, mir 1973; mir 1974; mir 1977, mir 1978, mir 1979, mir 4284, mir 4313, and mir 362-5p; or

mir 1974 and one or more of the following biomarkers: mir 16; mir 21; mir 15b; mir 191; mir 195; mir 652, mir 1308; mir 1915, mir 1973; mir 1977, mir 1978, mir 1979, mir 4284, mir 4313, and mir 362-Sp; or

mir 4284 and one or more of the following biomarkers: mir 16; mir 21; mir 15b; mir 191; mir 195; mir 652, mir 1308; mir 1915, mir 1973; mir 1974; mir 1977, mir 1978, mir 1979, mir 4284, mir 4313, and mir 362-5p; or

mir 15b and one or more of, or at least two of, mir 16, mir 191, mir 195, mir 1974, and mir 4284; or

mir 16 and one or more of, or at least two of, mir 15b, mir 191, mir 195, mir 1974, and mir 4284; or

mir 191 and one or more of, or at least two of, mir 15b, mir 16, mir 195, mir 1974, and mir 4284; or

mir 195 and one or more of, or at least two of mir 15b, mir 16, mir 191, mir 1974, and mir 4284; or

mir 1974 and one or more of, or at least two of, mir 15b, mir 16, mir 191, mir 195, and mir 4284; or

mir 4284 and one or more of, or at least two of, mir 15b, mir 16, mir 191, mir 195, and mir 1974; or

mir 16, alone or with one or both of mir 195 and mir 191; or

mir 195, alone or with one or both of mir 16 and mir 191; or

mir 191, alone or with one or both of mir 16 and mir 195; or

mir 16, mir 195, and mir 191, optionally with at least one or more of mir 1974, mir 4284, mir-15b, mir-1978, mir-1979, mir-362-5p and mir 1973; or

mir 16, mir 195, and mir 191, optionally with at least one or more of mir 1974, mir 4284 and mir-15b;

and comparing the level(s) with the level(s) of said biomarker(s) in a healthy control (either by measuring the level in a plasma or blood sample from one or more healthy individuals or by comparing to a predetermined reference value obtained using one or more healthy individuals), where an increase in the level(s) of the biomarker(s) in the subject relative to the level(s) in the control indicates that the subject has endometriosis, and if endometriosis is indicated, treating or recommending treating the subject with one or more of a pain medication, a hormone therapy (e.g., hormonal contraceptive, gonadotropin-releasing hormone (Gn-RH) agonists and antagonists, danazol, medroxyprogesterone, aromatase inhibitor) and/or surgery. In specific non-limiting embodiments, measurement of the level of miRNA may be performed by qPCR, Nanostring, microarray analysis, or next generation sequencing of RNA (eg miRNA) prepared from a plasma sample collected from the subject. In certain non-limiting embodiments, the diagnostic method may further comprise recommending or performing laparoscopic obtention of a tissue biopsy and/or recommending or performing a pelvic ultrasound to further support the diagnosis. In certain non-limiting embodiments laparoscopy and/or ultrasound may be performed if the plasma levels of the biomarker(s) is/are indicative of endometriosis.

In non-limiting embodiments, the present invention provides for a kit for determining whether a subject suffers from endometriosis comprising measurement means for:

one or more or two or more or three or more or four or more of the following biomarkers: mir 16; mir 21; mir 15b; mir 191; mir 195; mir 652, mir 1308; mir 1915, mir 1973; mir 1974; mir 1977, mir 1978, mir 1979, mir 4284, mir 4313, and mir 362-5p; or

one or more or two or more or three or more or four or more of the following biomarkers: mir 16; mir 15b; mir 191; mir 195; mir 1973; mir 1974; mir 1977, mir 1978, mir 1979, mir 4284 and mir 362-5p; or

mir 15b and one or more of the following biomarkers: mir 16; mir 21; mir 191; mir 195; mir 652, mir 1308; mir 1915, mir 1973; mir 1974; mir 1977, mir 1978, mir 1979, mir 4284, mir 4313 and mir 362-5p; or

mir 16 and one or more of the following biomarkers: mir 21; mir 15b; mir 191; mir 195; mir 652, mir 1308; mir 1915, mir 1973; mir 1974; mir 1977, mir 1978, mir 1979, mir 4284, mir 4313 and mir 362-5p; or

mir 191 and one or more of the following biomarkers; mir 16; mir 21; mir 15b; mir 195; mir 652, mir 1308; mir 1915, mir 1973; mir 1974; mir 1977, mir 1978, mir 1979, mir 4284, mir 4313, and mir 362-5p; or

mir 195 and one or more of the following biomarkers: mir 16; mir 21; mir 15b; mir 191; mir 652, mir 1308; mir 1915, mir 1973; mir 1974; mir 1977, mir 1978, mir 1979, mir 4284, mir 4313 and mir 362-5p; or

mir 1974 and one or more of the following biomarkers: mir 16; mir 21; mir 15b; mir 191; mir 195; mir 652, mir 1308; mir 1915, mir 1973; mir 1977, mir 1978, mir 1979, mir 4284, mir 4313, and mir 362-5p; or

mir 4284 and one or more of the following biomarkers: mir 16; mir 21; mir 15b; mir 191; mir 195; mir 652, mir 1308; mir 1915, mir 1973; mir 1974; mir 1977, mir 1978, mir 1979, mir 4284, mir 4313, and mir 362-5p; or

mir 15b and one or more of, or at least two of, mir 16, mir 191, mir 195, mir 1974, and mir 4284; or

mir 16 and one or more of, or at least two of, mir 15b, mir 191, mir 195, mir 1974, and mir 4284; or

mir 191 and one or more of, or at least two of, mir 15b, mir 16, mir 195, mir 1974, and mir 4284; or

mir 195 and one or more of, or at least two of mir 15b, mir 16, mir 191, mir 1974, and mir 4284; or

mir 1974 and one or more of, or at least two of, mir 15b, mir 16, mir 191, mir 195, and mir 4284; or

mir 4284 and one or more of, or at least two of, mir 15b, mir 16, mir 191, mir 195, and mir 1974; or

mir 16, alone or with one or both of mir 195 and mir 191; or

mir 195, alone or with one or both of mir 16 and mir 191; or

mir 191, alone or with one or both of mir 16 and mir 195; or

mir 16, mir 195, and mir 191, optionally with at least one or more of mir 1974, mir 4284, mir-15b, mir-1978, mir-1979, mir-362-5p and mir 1973; or

mir 16, mir 195, and mir 191, optionally with at least one or more of mir 1974, mir 4284 and mir-15b;

optionally together with a control (healthy) sample and/or a endometriosis sample.

In a specific, non-limiting embodiment, a kit may comprise a pair of oligonucleotide primers, suitable for polymerase chain reaction, for each miRNA to be measured.

In certain non-limiting embodiments, where the measurement means in the kit employs a microarray, the set of biomarkers set forth above may constitute at least 10 percent or at least 20 percent or at least 30 percent or at least 40 percent or at least 50 percent or at least 60 percent or at least 70 percent or at least 80 percent of the species of biomarkers represented on the microarray.

In certain non-limiting embodiments, where the kit includes means for measuring additional biomarkers, the set of biomarkers set forth above may constitute at least 10 percent or at least 20 percent or at least 30 percent or at least 40 percent or at least 50 percent or at least 60 percent or at least 70 percent or at least 80 percent of the species of biomarkers represented in the kit.

### 5.2 Plasma Biomarkers to Distinguish Between a Subject with Endometriosis and a Subject with Endometriosis-Associated Ovarian Cancer

The present invention provides for the following plasma mir biomarkers which differ between a subject with endometriosis and a subject with endometriosis-associated ovarian cancer (“EAOC”).

The present invention relates to a method of diagnosing EAOC in a subject, comprising measuring or having measured (i.e., directing measurement of) the plasma level of:

one or more or two or more or three or more or four or more of the following biomarkers: mir 21, mir 191, mir 744, mir 1308, mir 1975, mir 1977, mir 1274a, mir 766, mir 376a, mir 1246 and mir 362-5p; or

one or more or two or more or three or more or four or more of the following biomarkers: mir 362-5p, mir 1274a, mir-21, mir 766, mir 1975, mir 1308, mir 191, mir 744, mir 376a, and mir 1246; or

mir 362-5p and one or more of the following biomarkers: mir 21, mir 191, mir 744, mir 1308, mir 1975, and mir 1274a; or

mir 744 and one or more of the following biomarkers: mir 21, mir 191, mir 1308, mir 1975, mir 1274a and mir 362-5p; or

mir 362-5p and mir 1274a or mir 744; or

mir 362-5p and mir 1274a and mir 744; or

mir 362-5p alone or with one or both of mir 1274a and mir 21; or

mir 1274a alone or with one or both of mir 362-59 and mir 21; or

mir 21 alone or with one or both of mir 362-5p and mir 1274a; or

mir 362-5p, mir 1274a and mir 21, optionally with at least one or more of mir 766, mir 1975, mir 1308, mir 191, mir 744, mir 376a, and mir 1246; or mir 362-5p, mir 1274a and mir 21, optionally with at least one or more of mir 766, mir 1975 and mir 1308;

and comparing the level(s) with the level(s) of said biomarker(s) in a control subject with endometriosis (an “endometriosis control”; either by measuring the level in a plasma or blood sample from one or more subject with endometriosis or by comparing to a predetermined reference value obtained using one or more subjects having endometriosis), where a difference between the level(s) of the biomarker(s) in the subject relative to the level(s) in the control indicates that the subject has EAOC. In specific non-limiting embodiments, measurement of the level of miRNA may be performed by qPCR, Nanostring, microarray analysis, or next generation sequencing of RNA (eg miRNA) prepared from a plasma sample collected from the subject. In certain non-limiting embodiments, the diagnostic method may further comprise recommending or performing obtention of a tissue biopsy and/or recommending or performing a pelvic ultrasound or other imaging study (e.g. computer assisted tomography (CAT) scan, magnetic resonance imaging (MRI) or Positron Emmision Tomography (PET scan)) to further support the diagnosis.

In certain non-limiting embodiments, relative to an endometriosis control value, the plasma level in a subject with EAOC is increased for: mir 21, mir 191, mir 744, mir 1975, mir 766, mir 1246 and/or mir 376a, and/or is decreased for mir 1308, mir 1274a, and/or mir 362-5p Accordingly, in the methods described in this section, an increase in mir 21, mir 191, mir 744, mir 1975, mir 766, mir 1246 and/or mir 376a relative to an endometriosis control value indicates (supports a diagnosis of) EAOC in the subject and/or a decrease in mir 1308, mir 1274a, and/or mir 362-5p relative to an endometriosis control value indicates (supports a diagnosis of) EAOC in the subject.

The present invention provides for a method of treating a subject suffering from EAOC comprising diagnosing EAOC by measuring or having measured the plasma level of:

one or more or two or more or three or more or four or more of the following biomarkers: mir 21, mir 191, mir 744, mir 1308, mir 1975, mir 1274a, mir 766, mir 376a, mir 1246 and mir 362-5p; or

one or more or two or more or three or more or four or more of the following biomarkers: mir 362-5p, mir 1274a, mir-21, mir 766, mir 1975, mir 1308, mir 191, mir 744, mir 376a, and mir 1246; or

mir 362-5p and one or more of the following biomarkers: mir 21, mir 191, mir 744, mir 1308, mir 1975, and mir 1274a; or

mir 744 and one or more of the following biomarkers: mir 21, mir 191, mir 1308, mir 1975, mir 1274a and mir 362-5p; or

mir 362-5p and mir 1274a or mir 744; or

mir 362-5p and mir 1274a and mir 744; or

mir 362-5p alone or with one or both of mir 1274a and mir 21; or

mir 1274a alone or with one or both of mir 362-59 and mir 21; or

mir 21 alone or with one or both of mir 362-5p and mir 1274a; or

mir 362-5p, mir 1274a and mir 21, optionally with at least one or more of mir 766, mir 1975, mir 1308, mir 191, mir 744, mir 376a, and mir 1246; or

mir 362-5p, mir 1274a and mir 21, optionally with at least one or more of mir 766, mir 1975 and mir 1308;

and comparing the level(s) with the level(s) of said biomarker(s) in an endometriosis control (either by measuring the level in a plasma or blood sample from one or more subjects having endometriosis or by comparing to a predetermined reference value obtained using one or more subjects with endometriosis), where a difference between the level(s) of the biomarker(s) in the subject relative to the level(s) in the endometriosis control indicates that the subject has EAOC, and if EAOC is indicated treat or recommend treating the subject with one or more of a tissue biopsy, ovarectomy, hysterectomy, chemotherapy, and/or radiation therapy. In specific non-limiting embodiments, measurement of the level of miRNA may be performed by qPCR, Nanostring, microarray analysis, or next generation sequencing of RNA (eg miRNA) prepared from a plasma sample collected from the subject.

In non-limiting embodiments, the present invention provides for a kit for determining whether a subject suffers from EAOC comprising measurement means for:

one or more or two or more or three or more or four or more of the following biomarkers: mir 21, mir 191, mir 744, mir 1308, mir 1975, mir 1274a, mir 766, mir 376a, mir 1246 and mir 362-5p; or

one or more or two or more or three or more or four or more of the following biomarkers: mir 362-5p, mir 1274a, mir-21, mir 766, mir 1975, mir 1308, mir 191, mir 744, mir 376a, and mir 1246; or

mir 362-5p and one or more of the following biomarkers: mir 21, mir 191, mir 744, mir 1308, mir 1975, and mir 1274a; or

mir 744 and one or more of the following biomarkers: mir 21, mir 191, mir 1308, mir 1975, mir 1274a and mir 362-5p; or

mir 362-5p and mir 1274a or mir 744; or

mir 362-5p and mir 1274a and mir 744; or

mir 362-5p alone or with one or both of mir 1274a and mir 21; or

mir 1274a alone or with one or both of mir 362-59 and mir 21; or

mir 21 alone or with one or both of mir 362-5p and mir 1274a; or

mir 362-5p, mir 1274a and mir 21, optionally with at least one or more of mir 766, mir 1975, mir 1308, mir 191, mir 744, mir 376a, and mir 1246; or

mir 362-5p, mir 1274a and mir 21, optionally with at least one or more of mir 766, mir 1975 and mir 1308;

optionally together with a control (endometriosis) sample and/or an EAOC sample.

In a specific, non-limiting embodiment, a kit may comprise a pair of oligonucleotide primers, suitable for polymerase chain reaction, for each miRNA to be measured. Such primers may be designed based on the sequences for said miRNAs, which are known in the art.

In certain non-limiting embodiments, where the measurement means in the kit employs a microarray, the set of biomarkers set forth above may constitute at least 10 percent or at least 20 percent or at least 30 percent or at least 40 percent or at least 50 percent or at least 60 percent or at least 70 percent or at least 80 percent of the species of biomarkers represented on the microarray.

In certain non-limiting embodiments, where the kit includes means for measuring additional biomarkers, the set of biomarkers set forth above may constitute at least 10 percent or at least 20 percent or at least 30 percent or at least 40 percent or at least 50 percent or at least 60 percent or at least 70 percent or at least 80 percent of the species of biomarkers represented in the kit.

### 5.3 Plasma Biomarkers to Distinguish Between a Subject with Serous Ovarian Cancer and a Subject with Endometriosis Associated Ovarian Cancer

The present invention provides for the following plasma mir biomarkers which differ between a subject with serous ovarian cancer and a subject with endometriosis-associated ovarian cancer (“EAOC”).

The present invention relates to a method of diagnosing serous ovarian cancer or EAOC in a subject, comprising measuring or having measured (i.e., directing measurement of) the plasma level of:

one or more or two or more or three or more or four or more of the following biomarkers: mir 16, mir 21, mir 15b, mir 191, mir 652, mir 744, mir 1246, mir 1973, mir 1974, mir 1975, mir 1977, mir 1979, mir 376a, mir 195 and mir 362-5p; or

one or more or two or more or three or more or four or more of the following biomarkers: mir 21, mir 16, mir 191, mir 15b, mir 1975, mir 1246, mir 362-5p, mir 1979, mir 1973 and mir 195; or

mir 21 and one or more of the following biomarkers: mir 16, mir 15b, mir 191, mir 1246, mir 1973, mir 1975, mir 1979, mir 195 and mir 362-5p; or

mir 191 one or more of the following biomarkers: mir 16, mir 21, mir 15b, mir 1246, mir 1973, mir 1975, mir 1979, mir 195 and mir 362-5p; or

mir 362-5p and one or more of the following biomarkers: mir 16, mir 21, mir 15b, mir 191, mir 1246, mir 1973, mir 1975, mir 1979 and mir 195; or

mir 1979 and one or more of the following biomarkers: mir 16, mir 21, mir 15b, mir 191, mir 1246, mir 1973, mir 1975, mir 195 and mir 362-5p; or

mir 1975 and one or more of the following biomarkers: mir 16, mir 21, mir 15b, mir 191, mir 1246, mir 1973, mir 1975, mir 1979, mir 195 and mir 362-5p; or

mir 21 and one or more of, or at least two of, mir 191, mir 362-5p, mir 1979 and mir 1975; or

mir 191 and one or more of, or at least two of, mir 21, mir 362-5p, mir 1979 and mir 1975; or

mir 362-5p and one or more of, or at least two of, mir 21, mir 191, mir 1979 and mir 1975; or

mir 1979 and one or more of, or at least two of, mir 21, mir 191, mir 362-5p and mir 1975; or

mir 1975 and one or more of, or at least two of, mir 21, mir 191, mir 362-5p and mir 1979;

mir 21 alone or with one or both of mir 191 and mir 1975; or

mir 191 alone or with one or both of mir 21 and mir 1975; or

mir 1975 alone or with one or both of mir 21 and mir 191; or

mir 21, mir 191 and mir 1975, optionally with at least one or more of mir 16, mir 15b, mir 1246, mir 362-5p, mir 1979, mir 1973 and mir 195; or

mir 21, mir 191 and mir 1975, optionally with at least one or more of mir 16, mir 15b and mir 1246;

and comparing the level(s) with the level(s) of said biomarker(s) in a serous ovarian cancer control (either by measuring the level in a plasma or blood sample from one or more subjects having serous ovarian cancer or by comparing to a predetermined reference value obtained using one or more subjects having serous ovarian cancer), where a difference between the level(s) of the biomarker(s) in the subject relative to the level(s) in the serous ovarian cancer control indicates that the subject has EAOC. In specific non-limiting embodiments, measurement of the level of miRNA may be performed by qPCR, Nanostring, microarray analysis, or next generation sequencing of RNA (eg miRNA) prepared from a plasma sample collected from the subject. In certain non-limiting embodiments, the diagnostic method may further comprise recommending or performing obtention of a tissue biopsy and/or recommending or performing a pelvic ultrasound or other imaging study (e.g. computer assisted tomography (CAT) scan, magnetic resonance imaging (MRI) or Positron Emmision Tomography (PET scan)) to further support the diagnosis.

In certain non-limiting embodiments, relative to serous ovarian cancer control, the plasma level in a subject with EAOC is increased for: mir 16, mir 21, mir 15b, mir 191, mir 652, mir 744, mir 1246, mir 1973, mir 1974, mir 1975, mir 1977, mir 1979, mir 195 and mir 376a, and is decreased for mir 362-5p.

Accordingly, in the methods described in this section, an increase in mir 16, mir 21, mir 15b, mir 191, mir 652, mir 744, mir 1246, mir 1973, mir 1974, mir 1975, mir 195 and/or mir 1977 relative to a serous ovarian cancer control value indicates (supports a diagnosis of) EAOC in the subject and/or a decrease in mir 362-5p relative to a serous ovarian cancer control value indicates (supports a diagnosis of) EAOC in the subject.

The present invention provides for a method of treating a subject suffering from EAOC comprising diagnosing EAOC by measuring or having measured the plasma level of:

one or more or two or more or three or more or four or more of the following biomarkers: mir 16, mir 21, mir 15b, mir 191, mir 652, mir 744, mir 1246, mir 1973, mir 1974, mir 1975, mir 1977, mir 1979, mir 376a, mir 195 and mir 362-5p; or

one or more or two or more or three or more or four or more of the following biomarkers: mir 21, mir 16, mir 191, mir 15b, mir 1975, mir 1246, mir 362-5p, mir 1979, mir 1973 and mir 195; or

mir 21 and one or more of the following biomarkers: mir 16, mir 15b, mir 191, mir 1246, mir 1973, mir 1975, mir 1979 mir 195 and mir 362-5p; or

mir 191 one or more of the following biomarkers: mir 16, mir 21, mir 15b, mir 1246, mir 1973, mir 1975, mir 1979 mir 195 and mir 362-5p; or

mir 362-5p and one or more of the following biomarkers: mir 16, mir 21, mir 15b, mir 191, mir 1246, mir 1973, mir 1975, mir 195 and mir 1979; or

mir 1979 and one or more of the following biomarkers: mir 16, mir 21, mir 15b, mir 191, mir 1246, mir 1973, mir 1975, mir 195 and mir 362-5p; or

mir 1975 and one or more of the following biomarkers: mir 16, mir 21, mir 15b, mir 191, mir 1246, mir 1973, mir 1975, mir 1979, mir 195 and mir 362-5p; or

mir 21 and one or more of, or at least two of, mir 191, mir 362-5p, mir 1979 and mir 1975; or

mir 191 and one or more of, or at least two of, mir 21, mir 362-5p, mir 1979 and mir 1975; or

mir 362-5p and one or more of, or at least two of, mir 21, mir 191, mir 1979 and mir 1975; or

mir 1979 and one or more of, or at least two of, mir 21, mir 191, mir 362-Sp and mir 1975; or

mir 1975 and one or more of, or at least two of, mir 21, mir 191, mir 362-5p and mir 1979; or

mir 21 alone or with one or both of mir 191 and mir 1975; or

mir 191 alone or with one or both of mir 21 and mir 1975; or

mir 1975 alone or with one or both of mir 21 and mir 191; or

mir 21, mir 191 and mir 1975, optionally with at least one or more of mir 16, mir 15b, mir 1246, mir 362-5p, mir 1979, mir 1973 and mir 195; or

mir 21, mir 191 and mir 1975, optionally with at least one or more of mir 16, mir 15b and mir 1246;

and comparing the level(s) with the level(s) of said biomarker(s) in a control (either by measuring the level in a plasma or serum sample from one or more healthy individuals or by comparing to a predetermined reference value obtained using one or more healthy individuals), where a difference between the level(s) of the biomarker(s) in the subject relative to the level(s) in the control indicates that the subject has EAOC, and if EAOC is indicated treat or recommend treating the subject with one or more of a tissue biopsy, ovarectomy, hysterectomy, chemotherapy, and/or radiation therapy. In specific non-limiting embodiments, measurement of the level of miRNA may be performed by qPCR, Nanostring, microarray analysis, or next generation sequencing of RNA (eg miRNA) prepared from a plasma sample collected from the subject.

In non-limiting embodiments, the present invention provides for a kit for determining whether a subject suffers from EAOC comprising measurement means for:

one or more or two or more or three or more or four or more of the following biomarkers: mir 16, mir 21, mir 15b, mir 191, mir 652, mir 744, mir 1246, mir 1973, mir 1974, mir 1975, mir 1977, mir 1979, mir 376a, mir 195 and mir 362-5p; or

one or more or two or more or three or more or four or more of the following biomarkers: mir 21, mir 16, mir 191, mir 15b, mir 1975, mir 1246, mir 362-5p, mir 1979, mir 1973 and mir 195; or

mir 21 and one or more of the following biomarkers: mir 16, mir 15b, mir 191, mir 1246, mir 1973, mir 1975, mir 1979, mir 195 and mir 362-5p; or

mir 191 one or more of the following biomarkers: mir 16, mir 21, mir 15b, mir 1246, mir 1973, mir 1975, mir 1979, mir 195 and mir 362-5p; or

mir 362-5p and one or more of the following biomarkers: mir 16, mir 21, mir 15b, mir 191, mir 1246, mir 1973, mir 1975, mir 1979, and mir 195; or

mir 1979 and one or more of the following biomarkers: mir 16, mir 21, mir 15b, mir 191, mir 1246, mir 1973, mir 1975, mir 195 and mir 362-5p; or

mir 1975 and one or more of the following biomarkers: mir 16, mir 21, mir 15b, mir 191, mir 1246, mir 1973, mir 1975, mir 1979, mir 195 and mir 362-5p; or

mir 21 and one or more of, or at least two of, mir 191, mir 362-5p, mir 1979 and mir 1975; or

mir 191 and one or more of or at least two of, mir 21, mir 362-5p, mir 1979 and mir 1975; or

mir 362-5p and one or more of, or at least two of, mir 21, mir 191, mir 1979 and mir 1975; or

mir 1979 and one or more of, or at least two of, mir 21, mir 191, mir 362-5p and mir 1975; or

mir 1975 and one or more of, or at least two of, mir 21, mir 191, mir 362-5p and mir 1979; or

mir 21 alone or with one or both of mir 191 and mir 1975; or

mir 191 alone or with one or both of mir 21 and mir 1975; or

mir 1975 alone or with one or both of mir 21 and mir 191; or

mir 21, mir 191 and mir 1975, optionally with at least one or more of mir 16, mir 15b, mir 1246, mir 362-5p, mir 1979, mir 1973 and mir 195; or

mir 21, mir 191 and mir 1975, optionally with at least one or more of mir 16, mir 15b and mir 1246;

optionally together with a control (healthy) sample and/or a endometriosis sample and/or an EAOC sample.

In a specific, non-limiting embodiment, a kit may comprise a pair of oligonucleotide primers, suitable for polymerase chain reaction, for each miRNA to be measured. Such primers may be designed based on the sequences for said miRNAs, which are known in the art.

In certain non-limiting embodiments, where the measurement means in the kit employs a microarray, the set of biomarkers set forth above may constitute at least 10 percent or at least 20 percent or at least 30 percent or at least 40 percent or at least 50 percent or at least 60 percent or at least 70 percent or at least 80 percent of the species of biomarkers represented on the microarray.

In certain non-limiting embodiments, where the kit includes means for measuring additional biomarkers, the set of biomarkers set forth above may constitute at least 10 percent or at least 20 percent or at least 30 percent or at least 40 percent or at least 50 percent or at least 60 percent or at least 70 percent or at least 80 percent of the species of biomarkers represented in the kit.

### 5.4 Plasma Biomarkers to Distinguish Between a Healthy Subject and a Subject with Serous Ovarian Cancer

The present invention provides for the following plasma mir biomarkers which differ between a healthy subject and a subject with serous ovarian cancer.

The present invention relates to a method of diagnosing serous ovarian cancer in a subject, comprising measuring or having measured (i.e., directing measurement of) the plasma level of:

one or more or two or more or three or more or four or more of the following biomarkers: mir 16, mir 21, mir 15b, mir 191, mir 195, mir 1973, mir 1975, mir 1974, mir 1977, mir 1978, mir 1979, mir 4284, mir 4313 mir 1308 and mir 362-5p; or

one or more or two or more or three or more or four or more of the following biomarkers: mir 4284, mir 1974, mir 16, mir 1977, mir 1975, mir 195, mir 1978, mir 21, mir 362-5p, mir 15b, mir 1308 and mir 191;

mir 16 and one or more of the following biomarkers: mir 21, mir 15b, mir 191, mir 195, mir 1974, mir 1975, mir 1977, mir 1978, mir 4284, mir 1308 and mir 362-5p; or

mir 4284 and one or more of the following biomarkers: mir 16, mir 21, mir 15b, mir 191, mir 195, mir 1974, mir 1975, mir 1977, mir 1978, mir 1308 and mir 362-5p; or

mir 195 and one or more of the following biomarkers: mir 16, mir 21, mir 15b. mir 191, mir 1974, mir 1975, mir 1977, mir 1978, mir 4284, mir 1308 and mir 362-5p; or

mir 1974 and one or more of the following biomarkers: mir 16, mir 21, mir 15b, mir 191, mir 195, mir 1975, mir 1977, mir 1978, mir 1308 mir 4284 and mir 362-5p; or

mir 191 and one or more of the following biomarkers: mir 16, mir 21, mir 15b, mir 195, mir 1974, mir 1975, mir 1977, mir 1978, mir 4284, mir 1308 and mir 362-5p; or

mir 16 and one or more of, or at least two of, mir 4284, mir 195, mir 1974 and mir 191; or

mir 4284 and one or more of, or at least two of, mir 16, mir 195, mir 1974, and mir 191; or

mir 195 and one or more of, or at least two of, mir 16, mir 4284, mir 1974, and mir 191; or

mir 1974 and one or more of, or at least two of, mir 16, mir 4284, mir 195, and mir 191; or

mir 191 and one or more of, or at least two of, mir 16, mir 4284, mir 195, and mir 1974; or

mir 16, alone or with one or both of mir 191 and mir 4284; or

mir 191, alone or with one or both of mir 16 and mir 4284, or

mir 4284, alone or with one or both of mir 16 and mir 191; or

mir 16, mir 191 and mir 4284, optionally with at least one or more of mir 1974, mir 1977, mir 1975, mir 195, mir 1978, mir 21, mir 362-5p, mir 1308 and mir 15b; or

mir 16, mir 191 and mir 4284, optionally with at least one or more of mir 1974, mir 1977 and mir 1975;

and comparing the level(s) with the level(s) of said biomarker(s) in a healthy control (either by measuring the level in a plasma or blood sample from one or more healthy individuals or by comparing to a predetermined reference value obtained using one or more healthy individuals), where an increase in the level(s) of the biomarker(s) (or, in the case of mir 1975, a decrease) in the subject relative to the level(s) in the control indicates that the subject has serous ovarian cancer. In specific non-limiting embodiments, measurement of the level of miRNA may be performed by qPCR, Nanostring, microarray analysis, or next generation sequencing of RNA (eg miRNA) prepared from a plasma sample collected from the subject. In certain non-limiting embodiments, the diagnostic method may further comprise recommending or performing obtention of a tissue biopsy and/or recommending or performing a pelvic ultrasound or other imaging study (e.g. computer assisted tomography (CAT) scan, magnetic resonance imaging (MRI) or Positron Emmision Tomography (PET scan)) to further support the diagnosis.

In certain non-limiting embodiments, relative to healthy control, the plasma level in a subject with serous ovarian cancer is increased for: mir 16, mir 21, mir 15b, mir 191, mir 195, mir 1973, mir 1974, mir 1977, mir 1978, mir 1979. mir 4284, mir 4313, mir 1308 and mir 362-5p, and is decreased for mir 1975

The present invention provides for a method of treating a subject suffering from serous ovarian cancer comprising diagnosing serous ovarian cancer by measuring or having measured the plasma level of:

one or more or two or more or three or more or four or more of the 0.5 following biomarkers: mir 16, mir 21, mir 15b, mir 191, mir 195, mir 1973, mir 1974, mir 1975, mir 1977, mir 1978, mir 1979, mir 4284, mir 4313, mir 1308 and mir 362-5p; or

one or more or two or more or three or more or four or more of the following biomarkers: mir 4284, mir 1974, mir 16, mir 1977, mir 1975, mir 195, mir 1978, mir 21, mir 362-5p, mir 15b, mir 1308 and mir 191;

mir 16 and one or more of the following biomarkers: mir 21, mir 15b, mir 191, mir 195, mir 1974, mir 1975, mir 1977, mir 1978, mir 4284, mir 4313, mir 1308 and mir 362-5p; or

mir 4284 and one or more of the following biomarkers: mir 16, mir 21, mir 15b, mir 191, mir 195, mir 1974, mir 1975, mir 1977, mir 1978 and mir 362-5p; or

mir 195 and one or more of the following biomarkers: mir 16, mir 21, mir 15b, mir 191, mir 1974, mir 1975, mir 1977, mir 1978, mir 4284 and mir 362-5p; or

mir 1974 and one or more of the following biomarkers: mir 16, mir 21, mir 15b, mir 191, mir 195, mir 1975, mir 1977, mir 1978, mir 4284 and mir 362-5p; or

mir 191 and one or more of the following biomarkers: mir 16, mir 21, mir 15b, mir 195, mir 1974, mir 1975, mir 1977, mir 1978, mir 4284 and mir 362-5p; or

mir 16 and one or more of, or at least two of, mir 4284, mir 195, mir 1974 and mir 191; or

mir 4284 and one or more of, or at least two of, mir 16, mir 195, mir 1974, and mir 191; or

mir 195 and one or more of, or at least two of, mir 16, mir 4284, mir 1974, and mir 191; or

mir 1974 and one or more of, or at least two of, mir 16, mir 4284, mir 195, and mir 191; or

mir 191 and one or more of, or at least two of, mir 16, mir 4284, mir 195, and mir 1974; or

mir 16, alone or with one or both of mir 191 and mir 4284; or

mir 191, alone or with one or both of mir 16 and mir 4284, or

mir 4284, alone or with one or both of mir 16 and mir 191; or

mir 16, mir 191 and mir 4284, optionally with at least one or more of mir 1974, mir 1977, mir 1975, mir 195, mir 1978, mir 21, mir 362-5p, mir 1308 and mir 15b; or

mir 16, mir 191 and mir 4284, optionally with at least one or more of mir 1974, mir 1977 and mir 1975;

and comparing the level(s) with the level(s) of said biomarker(s) in a healthy control (either by measuring the level in a plasma or blood sample from one or more healthy individuals or by comparing to a predetermined reference value obtained using one or more healthy individuals), where an increase in the level(s) of the biomarker(s) (or, in the ease of mir 1975, a decrease) in the subject relative to the level(s) in the control indicates that the subject has serous ovarian cancer, and if serous ovarian cancer is indicated treat or recommend treating the subject with one or more of a tissue biopsy, ovarectomy, hysterectomy, chemotherapy, and/or radiation therapy. In specific non-limiting embodiments, measurement of the level of miRNA may be performed by qPCR, Nanostring, microarray analysis, or next generation sequencing of RNA (eg miRNA) prepared from a plasma sample collected from the subject.

In non-limiting embodiments, the method may further comprise measuring or having measured CA-125 levels, where if the miRNA level(s) indicates serous ovarian cancer, an elevated CA-125 level relative to health control corroborates the diagnosis of serous ovarian cancer.

In non-limiting embodiments, the present invention provides for a kit for determining whether a subject suffers from serous ovarian cancer comprising measurement means for:

one or more or two or more or three or more or four or more of the following biomarkers: mir 16, mir 21, mir 15b, mir 191, mir 195, mir 1973, mir 1974, mir 1975, mir 1977, mir 1978, mir 1979, mir 4284, mir 4313, mir 1308 and mir 362-5p; or

one or more or two or more or three or more or four or more of the following biomarkers: mir 4284, mir 1974, mir 16, mir 1977, mir 1975, mir 195, mir 1978, mir 21, mir 362-5p, mir 15b, mir 1308 and mir 191; or

mir 16 and one or more of the following biomarkers: mir 21, mir 15b, mir 191, mir 195, mir 1974, mir 1975, mir 1977, mir 1978, mir 4284, mir 1308 and mir 362-5p; or

mir 4284 and one or more of the following biomarkers: mir 16, mir 21, mir 15b, mir 191, mir 195, mir 1974, mir 1975, mir 1977, mir 1978, mir 1308 and mir 362-5p; or

mir 195 and one or more of the following biomarkers: mir 16, mir 21, mir 15b, mir 191, mir 1974, mir 1975, mir 1977, mir 1978, mir 4284, mir 1308 and mir 362-5p; or

mir 1974 and one or more of the following biomarkers: mir 16, mir 21, mir 15b, mir 191, mir 195, mir 1975, mir 1977, mir 1978, mir 4284, mir 1308 and mir 362-5p; or

mir 191 and one or more of the following biomarkers: mir 16, mir 21, mir 15b, mir 195, mir 1974, mir 1974, mir 1977, mir 1978, mir 4284, mir 1308 and mir 362-5p; or

mir 16 and one or more of, or at least two of, mir 4284, mir 195, mir 1974 and mir 191; or

mir 4284 and one or more of, or at least two of, mir 16, mir 195, mir 1974, and mir 191; or

mir 195 and one or more of, or at least two of, mir 16, mir 4284, mir 1974, and mir 191; or

mir 1974 and one or more of, or at least two of, mir 16, mir 4284, mir 195, and mir 191; or

mir 191 and one or more of, or at least two of, mir 16, mir 4284, mir 195, and mir 1974; or

mir 16, alone or with one or both of mir 191 and mir 4284; or

mir 191, alone or with one or both of mir 16 and mir 4284, or

mir 4284, alone or with one or both of mir 16 and mir 191; or

mir 16, mir 191 and mir 4284, optionally with at least one or more of mir 1974, mir 1977, mir 1975, mir 195, mir 1978, mir 21, mir 362-5p, mir 1308 and mir 15b; or

mir 16, mir 191 and mir 4284, optionally with at least one or more of mir 1974, mir 1977 and mir 1975;

optionally together with a control (healthy) sample and/or a serous ovarian cancer sample.

In a specific, non-limiting embodiment, a kit may comprise a pair of oligonucleotide primers, suitable for polymerase chain reaction, for each miRNA to be measured. Such primers may be designed based on the sequences for said miRNAs, which are known in the art.

In certain non-limiting embodiments, where the measurement means in the kit employs a microarray, the set of markers set forth above may constitute at least 10 percent or at least 20 percent or at least 30 percent or at least 40 percent or at least 50 percent or at least 60 percent or at least 70 percent or at least 80 percent of the species of markers represented on the microarray.

### 5.5 Plasma Biomarkers to Distinguish Between a Healthy Subject and a Subject with Endometriosis-Associated Ovarian Cancer

The present invention provides for the following plasma mir biomarkers which differ between a healthy subject and a subject with endometriosis-associated ovarian cancer (“EAOC”).

The present invention relates to a method of diagnosing EAOC in a subject, comprising measuring or having measured (i.e., directing measurement of) the plasma level of:

one or more or two or more or three or more or four or more of the following biomarkers: mir 16, mir 21, mir 15b, mir 191, mir 195, mir 652, mir 744, mir 1973, mir 1974, mir 1975, mir 1977, mir 1978, mir 1979, mir 4284, mir 766 and mir 376a; or

one or more or two or more or three or more or four or more of the following biomarkers: mir 16, mir 21, mir 15b, mir 191, mir 195, mir 1973, mir 1974, mir 1977, mir 1979, mir 766 and mir 4284; or

mir 21 and one or more of the following biomarkers: mir 16, mir 15b, mir 191, mir 195, mir 1973, mir 1974, mir 1977, mir 1979, mir 766 and mir 4284; or

mir 191 and one or more of the following biomarkers: mir 16, mir 21, mir 15b, mir 191, mir 195, mir 1973, mir 1974, mir 1977, mir 1979, mir 766 and mir 4284; or

mir 21 and mir 191; or

mir 16, alone or with one or both of mir 21 and mir 191; or

mir 21, alone or with one or both of mir 16 and mir 191; or

mir 191, alone or with one or both of mir 16 and mir 21; or

mir 16, mir 21, and mir 191, optionally with at least one or more of mir 15b, mir 195, mir 1973, mir 1974, mir 1977, mir 1979, mir 766 and mir 4284; or

mir 16, mir 21 and mir 191, optionally with at least one or more of mir 15b, mir 1977 and mir 1979;

and comparing the level(s) with the level(s) of said biomarker(s) in a healthy control (either by measuring the level in a plasma or blood sample from one or more healthy individuals or by comparing to a predetermined reference value obtained using one or more healthy individuals), where an increase in the level(s) of the biomarker(s) in the subject relative to the level(s) in the control indicates that the subject has EAOC. In specific non-limiting embodiments, measurement of the level of miRNA may be performed by OCR or microarray analysis of RNA (eg miRNA) prepared from a plasma sample collected from the subject. In certain non-limiting embodiments, the diagnostic method may further comprise recommending or performing obtention of a tissue biopsy and/or recommending or performing a pelvic ultrasound or other imaging study (e.g. computer assisted tomography (CAT) scan, magnetic resonance imaging (MRI) or Positron Emmision Tomography (PET scan)) to further support the diagnosis.

In certain non-limiting embodiments, relative to healthy control, the plasma level in a subject with EAOC is increased for: mir 16, mir 21, mir 15b, mir 191, mir 195, mir 652, mir 744, mir 1973, mir 1974, mir 1975, mir 1977, mir 1978, mir 1979, mir 4284, mir 766 and mir 376a.

The present invention provides for a method of treating a subject suffering from EAOC comprising diagnosing EAOC by measuring or having measured the plasma level of:

one or more or two or more or three or more or four or more of the following biomarkers: mir 16, mir 21, mir 15b, mir 191, mir 195, mir 652, mir 744, mir 1973, mir 1974, mir 1975, mir 1977, mir 1978, mir 1979, mir 4284, mir 766 and mir 376a; or

one or more or two or more or three or more or four or more of the following biomarkers: mir 16, mir 21, mir 15b, mir 191, mir 195, mir 1973, mir 1974, mir 1977, mir 1979, mir 766 and mir 4284; or

mir 21 and one or more of the following biomarkers: mir 16, mir 15b, mir 191, mir 195, mir 1973, mir 1974, mir 1977, mir 1979, mir 766 and mir 4284; or

mir 191 and one or more of the following biomarkers: mir 16, mir 21, mir 15b, mir 191, mir 195, mir 1973, mir 1974, mir 1977, mir 1979, mir 766 and mir 4284; or

mir 21 and mir 191; or

mir 16, alone or with one or both of mir 21 and mir 191; or

mir 21, alone or with one or both of mir 16 and mir 191; or

mir 191, alone or with one or both of mir 16 and mir 21; or

mir 16, mir 21, and mir 191, optionally with at least one or more of mir 15b, mir 195, mir 1973, mir 1974, mir 1977, mir 1979, mir 766 and mir 4284; or

mir 16, mir 21 and mir 191, optionally with at least one or more of mir 15b, mir 1977 and mir 1979;

and comparing the level(s) with the level(s) of said biomarker(s) in a healthy control (either by measuring the level in a plasma or blood sample from one or more healthy individuals or by comparing to a predetermined reference value obtained using one or more healthy individuals), where an increase in the level(s) of the biomarker(s) in the subject relative to the level(s) in the control indicates that the subject has EAOC, and if EAOC is indicated treat or recommend treating the subject with one or more of a tissue biopsy, ovarectomy, hysterectomy, chemotherapy, and/or radiation therapy. In specific non-limiting embodiments, measurement of the level of miRNA may be performed by qPCR, Nanostring, microarray analysis, or next generation sequencing of RNA (eg miRNA) prepared from a plasma sample collected from the subject.

In non-limiting embodiments, the present invention provides for a kit for determining whether a subject suffers from EAOC comprising measurement means for:

one or more or two or more or three or more or four or more of the following biomarkers: mir 16, mir 21, mir 15b, mir 191, mir 195, mir 652, mir 744, mir 1973, mir 1974, mir 1975, mir 1977, mir 1978, mir 1979, mir 4284, mir 766 and mir 376a; or

one or more or two or more or three or more or four or more of the following biomarkers: mir 16, mir 21, mir 15b, mir 191, mir 195, mir 1973, mir 1974, mir 1977, mir 1979, mir 766 and mir 4284; or

mir 21 and one or more of the following biomarkers: mir 16, mir 15b, mir 191, mir 195, mir 1973, mir 1974, mir 1977, mir 1979 and mir 4284; or

mir 191 and one or more of the following biomarkers: mir 16, mir 21, mir 15b, mir 191, mir 195, mir 1973, mir 1974, mir 1977, mir 1979, mir 766 and mir 4284; or

mir 21 and mir 191; or

mir 16, alone or with one or both of mir 21 and mir 191; or

mir 21, alone or with one or both of mir 16 and mir 191; or

mir 191, alone or with one or both of mir 16 and mir 21; or

mir 16, mir 21, and mir 191, optionally with at least one or more of mir 15b, mir 195, mir 1973, mir 1974, mir 1977, mir 1979, mir 766 and mir 4284; or

mir 16, mir 21 and mir 191, optionally with at least one or more of mir 15b, mir 1977 and mir 1979;

optionally together with a control (healthy) sample and/or an EAOC sample.

In a specific, non-limiting embodiment, a kit may comprise a pair of oligonucleotide primers, suitable for polymerase chain reaction, for each miRNA to be measured. Such primers may be designed based on the sequences for said miRNAs, which are known in the art.

In certain non-limiting embodiments, where the measurement means in the kit employs a microarray, the set of biomarkers set forth above may constitute at least 10 percent or at least 20 percent or at least 30 percent or at least 40 percent or at least 50 percent or at least 60 percent or at least 70 percent or at least 80 percent of the species of biomarkers represented on the microarray.

In certain non-limiting embodiments, where the kit includes means for measuring additional biomarkers, the set of biomarkers set forth above may constitute at least 10 percent or at least 20 percent or at least 30 percent or at least 40 percent or at least 50 percent or at least 60 percent or at least 70 percent or at least 80 percent of the species of biomarkers represented in the kit.

### 5.6 Plasma Biomarkers to Distinguish Between a Subject with Endometriosis and a Subject with Serous Ovarian Cancer

The present invention provides for the following plasma mir biomarkers which differ between a subject having endometriosis and a subject with serous ovarian cancer.

The present invention relates to a method of diagnosing serous ovarian cancer in a subject, comprising measuring or having measured (i.e., directing measurement of) the plasma level of:

one or more or two or more or three or more or four or more of the following biomarkers: mir 15b, mir 191, mir 362-5p, mir 628-3p, mir 1915, mir 1973, mir 362-5p, mir 16, mir 21, mir 195, mir 1308, mir 1974 and mir 652; or

mir 362-5p, alone or with one or both of mir 628-3p and mir 1915; or

mir 628-3p, alone or with one or both of mir 362-5p and mir 1915; or

mir 1915, alone or with one or both of mir 362-5p and mir 628-3p; or

mir 362-5p, mir 628-3p and mir 1915, optionally with at least one or more of mir 15B, mir 191, mir 362-5p, mir 628-3p, mir 1915, mir 1973, mir 362-5P, mir 16, mir 21, mir 195, mir 1308, mir 1974 and mir 652;

mir-1915 and/or mir 1274b and/or mir 362-5p, for example mir 1274b and mir 362-5p;

and comparing the level(s) with the level(s) of said biomarker(s) in an endometriosis control (either by measuring the level in a plasma or blood sample from one or more individuals having endometriosis or by comparing to a predetermined reference value obtained using one or more individuals having endometriosis), where a decrease in the level(s) of the biomarker(s) in the subject relative to the level(s) in the endometriosis control indicates that the subject has serous ovarian cancer. In specific non-limiting embodiments, measurement of the level of miRNA may be performed by qPCR, Nanostring, microarray analysis, or next generation sequencing of RNA (eg miRNA) prepared from a plasma sample collected from the subject. In certain non-limiting embodiments, the diagnostic method may further comprise recommending or performing obtention of a tissue biopsy and/or recommending or performing a pelvic ultrasound to further support the diagnosis.

In certain non-limiting embodiments, relative to an endometriosis control, the plasma level in a subject with serous ovarian cancer decreased for: mir 15b, mir 191, mir 1973, mir 16, mir 21, mir 195, mir 1308, mir 1974, mir 652, mir 1915, mir 628-3p and/or mir 362-5p.

The present invention provides for a method of treating a subject suffering from serous ovarian cancer comprising diagnosing serous ovarian cancer by measuring or having measured the plasma level of:

one or more or two or more or three or more or four or more of the following biomarkers: mir 15b, mir 191, mir 362-5p, mir 628-3p, mir 1915, mir 1973, mir 362-5P, mir 16, mir 21, mir 195, mir 1308, mir 1974 and mir 652; or

mir 362-5p, alone or with one or both of mir 628-3p and mir 1915; or

mir 628-3p, alone or with one or both of mir 362-5p and mir 1915; or

mir 1915, alone or with one or both of mir 362-5p and mir 628-3p; or

mir 362-5p, mir 628-3p and mir 1915, optionally with at least one or more of mir 15B, mir 191, mir 362-5p, mir 628-3p, mir 1915, mir 1973, mir 362-5P, mir 16, mir 21, mir 195, mir 1308, mir 1974 and mir 652;

mir-1915 and/or mir 1274b and/or mir 362-5p, for example mir 1274b and mir 362-5p;

and comparing the level(s) with the level(s) of said biomarker(s) in an endometriosis control (either by measuring the level in a plasma or blood sample from one or more individuals with endometriosis or by comparing to a predetermined reference value obtained using one or more individuals having endometriosis), where a decrease in the level(s) of the biomarker(s) in the subject relative to the level(s) in the control indicates that the subject has serous ovarian cancer, and if serous ovarian cancer is indicated treat or recommend treating the subject with one or more of a tissue biopsy, ovarectomy, hysterectomy, chemotherapy, and/or radiation therapy. In specific non-limiting embodiments, measurement of the level of miRNA may be performed by qPCR, Nanostring, microarray analysis, or next generation sequencing of RNA (eg miRNA) prepared from a plasma sample collected from the subject.

In non-limiting embodiments, the method may further comprise measuring or having measured CA-125 levels, where if miRNA levels indicates serous ovarian cancer, an elevated CA-125 level corroborates the diagnosis of serous ovarian cancer.

In non-limiting embodiments, the present invention provides for a kit for determining whether a subject suffers from serous ovarian cancer comprising measurement means for:

mir-1915 and/or mir 1274b and/or mir 362-5p, for example mir 1274b and mir 362-5p;

one or more or two or more or three or more or four or more of the following biomarkers: mir 15B, mir 191, mir 362-5p, mir 628-3p, mir 1915, mir 1973, mir 362-5P, mir 16, mir 21, mir 195, mir 1308, mir 1974 and mir 652; or

mir 362-5p, alone or with one or both of mir 628-3p and mir 1915; or

mir 628-3p, alone or with one or both of mir 362-5p and mir 1915; or

mir 1915, alone or with one or both of mir 362-5p and mir 628-3p; or

mir 362-5p, mir 628-3p and mir 1915, optionally with at least one or more of mir 15B, mir 191, mir 362-5p, mir 628-3p, mir 1915, mir 1973, mir 362-5P, mir 16, mir 21, mir 195, mir 1308, mir 1974 and mir 652;

optionally together with a control (healthy) sample and/or an endometriosis sample and/or a serous ovarian cancer sample.

In a specific, non-limiting embodiment, a kit may comprise a pair of oligonucleotide primers, suitable for polymerase chain reaction, for each miRNA to be measured. Such primers may be designed based on the sequences for said miRNAs, which are known in the art.

In certain non-limiting embodiments, where the measurement means in the kit employs a microarray, the set of biomarkers set forth above may constitute at least 10 percent or at least 20 percent or at least 30 percent or at least 40 percent or at least 50 percent or at least 60 percent or at least 70 percent or at least 80 percent of the species of biomarkers represented on the microarray.

In certain non-limiting embodiments, where the kit includes means for measuring additional biomarkers, the set of biomarkers set forth above may constitute at least 10 percent or at least 20 percent or at least 30 percent or at least 40 percent or at least 50 percent or at least 60 percent or at least 70 percent or at least 80 percent of the species of biomarkers represented in the kit.

### 5.7 Correlative Kits and Methods

In certain non-limiting embodiments, the present invention provides for methods and kits that permit a clinician to, using plasma miRNA biomarkers, assess the likelihood of whether a female subject has endometriosis, EAOC, or serous ovarian cancer. The methods of distinguishing between these conditions are set forth in the sections above.

A kit which may be used to distinguish between these conditions may comprise measuring means for:

one or more or two or more or three or more or four or more or five or more or six or more or seven or more or eight or more or nine or ten or eleven of mir 15b, mir 16, mir 21, mir 191, mir 195, mir 4284, mir 362-5p, mir 1274a, mir 1975, mir 628-3p, and mir 1915; or

mir 16, mir 21 and mir 191 and one or two or more or three or more or four or more or five or more or six or more or seven or eight of mir 15b, mir 195, mir 4284, mir 362-5p, mir 1274a, mir 1975, mir 628-3p, and mir 1915; or

mir 16, mir 191, and one, two or three of mir 21, mir 195, 4284;

mir 16, mir 21, and mir 191 and one or two of mir 4284 and mir 1975;

one or more of mir 16, mir 21, mir 15b, mir 191, mir 195, mir 652, mir 1308, mir 1915, mir 1973, mir 1974 and mir 1977, and preferably the panel, mir 16, mir 15b, mir 195, mir 4284, mir 191 and mir 1974;

one or more of mir 21, mir 191, mir 744, mir 1308, mir 1975, mir 1977, mir 1274a, mir 1274b, and mir 362-5p, and preferably the panel, mir 362, mir 1274b and mir 744;

one or more of mir 16, mir 21, mir 15b, mir 191, mir 652, mir 744, mir 1246, mir 1973, mir 1974, mir 1975, mir 1977, mir 1979, mir 376a and mir 362-5p, and preferably the panel, mir 21, mir 191, mir 362-5p, mir 1979 and mir 1975;

one or more of mir 16, mir 21, mir 15b, mir 191, mir 195, mir 1973, mir 1974, mir 1977, mir 1978, mir 1979, mir 4284, mir 4313 and mir 362-5p, and preferably the panel, mir 16, mir 4284, mir 1979, mir 1974 and mir 191;

one of mir 16, mir 21, mir 15b, mir 191, mir 195, mir 652, mir 744, mir 1973, mir 1974, mir 1975, mir 1977, mir 1978, mir 1979, mir 4284, and mir 376a, and preferably the panel, mir 21 and mir 191; and

one or more of mir 1915, mir 1274b and mir 362-5p, and preferably the panel, mir 362-5p and mir 1274b.

In a specific, non-limiting embodiment, a kit may comprise measuring means for:

mir 16, mir 15b, mir 195, mir 4284, mir 191, mir 1974, mir 362-5p, mir 1274b, mir 744, mir 21, mir 1979 and mir 1975.

In a specific, non-limiting embodiment, a kit may comprise a pair of oligonucleotide primers, suitable for polymerase chain reaction, for each miRNA to be measured. Such primers may be designed based on the sequences for said miRNAs, which are known in the art.

Any of the foregoing kits, in this or the preceding sections, may further optionally comprise one or more controls such as a healthy control, an endometriosis control, an EAOC control, and/or a serous ovarian cancer control. In non-limiting examples, such controls may be plasma samples or may be combinations of microRNAs prepared to resemble such natural plasma samples.

In certain non-limiting embodiments, the present invention provides for a method of diagnosing a subject, comprising (a) obtaining a plasma sample from the subject; (b) purifying nucleic acid from the sample; (c) amplifying, from the nucleic acid, one or more microRNA biomarker that distinguishes between a healthy subject and (i) a subject having endometriosis; (ii) a subject with endometriosis associated ovarian cancer; and (iii) a subject having serous ovarian cancer; (d) measuring the level(s) of the one or more microRNA biomarker amplified according to step (c); (e) comparing the level of the one or more biomarker in the plasma sample to a control level or control levels; and (f) reporting if a diagnosis of endometriosis, endometriosis associated ovarian cancer or serous ovarian cancer is indicated.

In certain non-limiting embodiments, the present invention provides for a method of evaluating whether a subject having endometriosis has developed ovarian cancer, comprising (a) obtaining a plasma sample from the subject; (b) purifying nucleic acid from the sample; (c) amplifying, from the nucleic acid, one or more microRNA biomarker that distinguishes between a subject with endometriosis and (i) a subject with endometriosis associated ovarian cancer and/or (ii) a subject with serous ovarian cancer; (d) measuring the level(s) of the one or more microRNA amplified according to step (e); (e) comparing the level of the one or more biomarker in the plasma sample to a control level or control levels; and (f) reporting if a diagnosis of endometriosis associated ovarian cancer or serous ovarian cancer is indicated.

In certain non-limiting embodiments, the present invention provides for a method of diagnosing a subject, comprising measuring, in a blood sample from the subject, the plasma level of biomarkers that distinguish between (a) a healthy subject and (i) a subject having endometriosis, (ii) a subject with EAOC; and (iii) a subject having serous ovarian cancer; and optionally also measuring or having measured the plasma level of biomarkers that distinguish between (b) a subject with endometriosis and (i) a subject with EAOC and/or (ii) a subject with serous ovarian cancer; and then, reporting if a diagnosis of endometriosis, EAOC or serous ovarian cancer is indicated. The method may optionally further comprise recommending or performing obtention of a tissue biopsy or an imaging study such as ultrasound (e.g pelvic ultrasound), computer assisted tomography (CAT) scan, magnetic resonance imaging (MRI) or Positron Emmision Tomography (PET scan).

In certain non-limiting embodiments, the present invention provides for a method of treating a subject, comprising measuring or having measured (i.e., directing measurement of), in a blood sample from the subject, the plasma level of biomarkers that distinguish between (a) a healthy subject and (i) a subject having endometriosis, (ii) a subject with EAOC; and (iii) a subject having serous ovarian cancer; and optionally also measuring or having measured the plasma level of biomarkers that distinguish between (b) a subject with endometriosis and (i) a subject with EAOC and/or (ii) a subject with serous ovarian cancer; and then, if a diagnosis of EAOC or serous ovarian cancer is indicated, treat or recommend treating the subject with one or more of a tissue biopsy, ovarectomy, hysterectomy, chemotherapy, and/or radiation therapy.

In certain non-limiting embodiments, the present invention provides for a method of evaluating whether a subject having endometriosis has developed ovarian cancer, comprising measuring, in a blood sample from the subject, the plasma level of biomarkers that distinguish between a subject with endometriosis and (i) a subject with EAOC and/or (ii) a subject with serous ovarian cancer; and then, reporting if a diagnosis of EAOC or serous ovarian cancer is indicated.

In certain non-limiting embodiments, the present invention provides for a method of treating (or monitoring) a subject suffering from endometriosis comprising measuring or having measured (i.e., directing measurement of), in a blood sample from the subject, the plasma level of biomarkers that distinguish between a subject with endometriosis and (i) a subject with EAOC and/or (ii) a subject with serous ovarian cancer; and then, if a diagnosis of EAOC or serous ovarian cancer is indicated, treat or recommend treating the subject with one or more of a tissue biopsy, ovarectomy, hysterectomy, chemotherapy, and/or radiation therapy.

In certain non-limiting embodiments, where the measurement means in the kit employs a microarray, the set of biomarkers set forth above may constitute at least 10 percent or at least 20 percent or at least 30 percent or at least 40 percent or at least 50 percent or at least 60 percent or at least 70 percent or at least 80 percent of the species of biomarkers represented on the microarray.

In certain non-limiting embodiments, where the kit includes means for measuring additional biomarkers, the set of biomarkers set forth above may constitute at least 10 percent or at least 20 percent or at least 30 percent or at least 40 percent or at least 50 percent or at least 60 percent or at least 70 percent or at least 80 percent of the species of biomarkers represented in the kit.

### 6. EXAMPLE 1

### Plasma MicroRNAs as Novel Biomarkers for Endometriosis and Endometriosis-Associated Ovarian Cancer

### 6.1 Materials and Methods

Ethics Statement.

This research study protocol was approved by the institutional review board (IRB) at the University of Pittsburgh, and informed consent was obtained from all study participants prior to blood collection.

Patients.

The clinical and demographic characteristics of patients with endometriosis (n=33), EAOC (n=14, of which six were endometrioid, seven were clear cell tumors and one had mixed endornetrioid/clear cell histology), and serous ovarian cancer (SOC, n=21) are shown in Table 2. All cancer patients were treated at Magee-Womens Hospital of the University of Pittsburgh Medical Center (UPMC) between 2006-2011. The inclusion criteria consisted of patients with primary ovarian tumors and confirmed histology of EAOC or SOC.

In the EAOC group, five patients had endometriosis on the final pathology. Of the five, two had endometrioid, two had clear cell tumors, and one had mixed histology of clear cell and endometrioid type.

The endometriosis patients were treated at Magee-Womens Hospital of UPMC between 2006-2011. Samples from patients with confirmed histology of endometriosis were included in this study. The cases where endometriosis could not be histologically confirmed on surgically removed tissues were excluded from this study.

Plasma samples from healthy women (controls, n=20) were purchased from Innovative Research Labs (Seattle, Wash.). Women without any current clinical conditions and without family history of diseases such as cancer, HIV, diabetes, and autoimmune diseases, were qualified as healthy individuals by Innovative Research Labs,

Specimen Characteristics.

Peripheral blood was drawn in heparinized tubes (BD Biosciences, San Jose, Calif.) and processed at Magee-Womens Research Institute within eight hours from collection. The tubes were centrifuged at 2,300 rpm for 20 minutes at room temperature. Plasma was collected in a sterile biohazard cabinet, aliquoted, and cryopreserved at −80° C. until ready to use. Processing of blood by Innovative Research Labs was similarly performed.

Study Design.

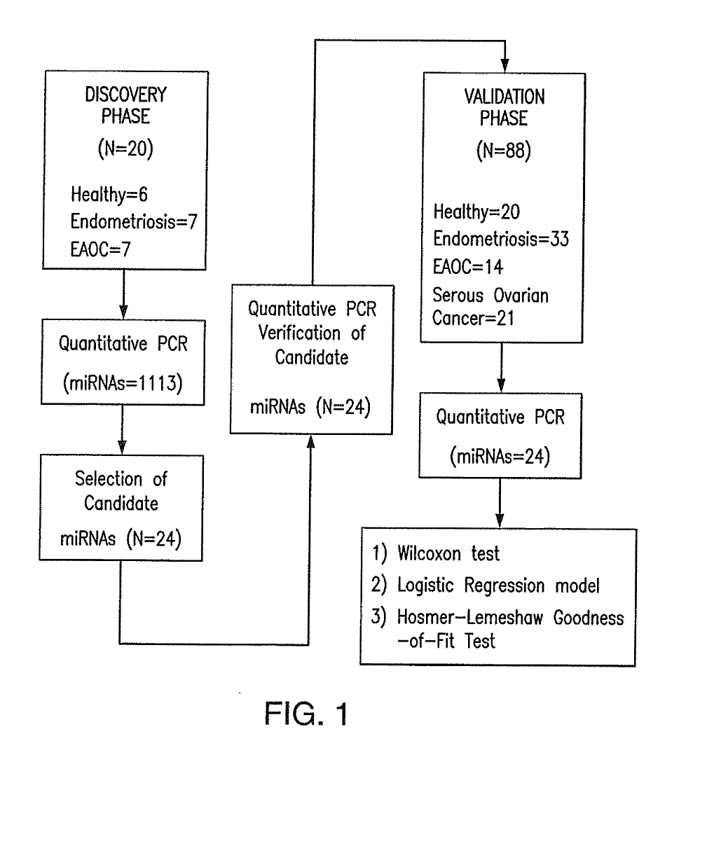

Of the total 88 retrospectively collected plasma samples, 20 were randomly selected for the initial discovery phase (healthy controls, n=6; endometriosis, n=7; EAOC, n=7, of which four were endometrioid and three were clear cell tumors). We used all of the 88 samples for our validation analyses. Study design is illustrated in FIG. 1. In the discovery phase, miRNAs extracted from 20 plasma samples were used to quantify a total of 1113 miRNAs by RT-qPCR. Expression of 23 candidate miRNAs that are differentially expressed in these three categories of samples and expression of an endogenous control miRNA, miR-132, were confirmed by an independent RT-qPCR in these 20 samples. Finally, expression of the 24 miRNAs was further studied in the complete cohort of 88 plasma samples, by RT-qPCR.

miRNA Isolation and RT-Qpcr Assay.

RNA was isolated from 88 plasma samples using the mirVana miRNA Isolation Kit (Life Technologies, Carlsbad, Calif.). Concentrations of RNA were measured by a NanoDrop 2000 spectrophotometer (Thermo Scientific, Wilmington, Del.). Sixty nanograms of purified RNA were used for RT using the QuantiMir Kit (System Biosciences, Mountain View, Calif.). One microliter of cDNA was then diluted 1:160 and 1.10 of diluted cDNA was used in each qPCR reaction for a genome-wide expression profiling of 1113 miRNAs (Sanger miRBase Version 15) using the Human miRNome Profiler kit (System Biosciences). qPCR was performed on an ABI7900HT Real-Time PCR System (Applied Biosystems, Foster City, Calif.) using the RT2 SYBR Green ROX qPCR master mix (Qiagen, Valencia, Calif.) under the following conditions: 50° C. for 2 minutes, 95° C. for 10 minutes, 40 cycles of 95° C. for 15 seconds followed by 60° C. for 10 seconds, and a standard dissociation stage. The RT-qPCR data were analyzed according to the comparative CT method (24).

NanoString nCounter miRNA Assay.

The miRNeasy FFPE Kit (Qiagen) was used to isolate miRNAs from formalin fixed paraffin embedded (FFPE) tissues for global miRNA profiling using matching plasma-tissue of endometriosis or EAOC samples. The nCounter Human miRNA Panel v2 that evaluates 800 miRNAs was used (NanoString, Seattle, Wash.). miRNAs extracted from plasma and tissue samples were subjected to nCounter miRNA sample preparation according to the manufacturer's instructions. This was followed by ligation of 100 ng of miRNA and hybridization to probes at 65° C. for 18 hours following the manufacturer's protocol. Next day, the hybridized probes were purified and counted on nCounter Prep Station and Digital Analyzer. The data obtained from Analyzer contained counts of individual fluorescent barcodes and thus, a count of miRNAs present in the sample. The nCounter results were analyzed by the nSolver software according to the manufacturer's instructions.

Plasma miRNA Measurements in LSL-KrasG12D/+/Ptenloxp/Loxp Mice with Endometrioid Ovarian Cancer.

All animal experiments were performed according to a protocol approved by the University of Pittsburgh International Animal Care and Use Committee (IACUC). The mice were originally provided by Dr. Dinulescu (25), and the colony was maintained at the Magee-Womens Research Institute. Genotyping for the identification of LSL-KrasG12D/+/Ptenloxp/loxp mice was performed as previously described (25).

Survival surgery procedure and administration of recombinant adenovirus encoding for Cre recombinase [Ad5CMVCre (AdCre)] (University of Iowa Gene Transfer Vector Core) was performed in synchronized animals as previously described by us and others (25, 26). Briefly, seven to nine weeks old KrasG12D/+/Ptenloxp/loxp virgin females were injected i.p. with 5 U pregnant mare's serum gonadotropin (PMSG) followed by 5 U human chorionic gonadotropin (hCG) 48 hours later. Thirty-six hours later, mice received 5 μl of 2.5×10⁷ plaque-forming units (p.f.u.) Ad5CMVCre delivered to the ovary surface epithelium (OSE) of the left ovary only, via intrabursal injection. The contra-lateral ovary served as a control.

Mice were sacrificed when disease was clinically evident (tumor mass on the injected side and/or ascites accumulation) or when mice were moribund (hunched appearance, ruffled fur, unable to reach for food or water). Blood was collected by cardiac puncture at necropsy and serum cryopreserved until ready to use. Expression of mouse miRNAs, mmu-miR-15b, 16, 21, 191, and 195 were measured by RT-qPCR as described above.

Statistical Analysis and Sample Size Justification.

The candidate demographic variables analyzed were: age, race, history of alcohol and tobacco consumption. We also compared stage of disease in EAOC versus SOC. For the baseline characteristics, we conducted univariate comparisons using Chi-square tests, ANOVA test, or their nonparametric equivalents, as appropriate. We determined the medians and interquartile range as measures of central tendency for variables with highly skewed distribution (not normally distributed such as gravidity or parity). All analyses were performed using SAS 9.3 (SAS Institute, Cary, N.C.) assuming statistical significance at p<0.05.

All qPCR CT values were normalized to miR-132. The ΔCT of sample i marker j is defined as ΔCTᵢⱼ=CTᵢⱼ−CTᵢₘᵢᵣ₁₃₂. Here CTᵢⱼ and CTᵢₘᵢᵣ₁₃₂ are CT values of marker j and miR-132 for sample i, respectively. The fold change (FC) between two specific groups for a specific marker was calculated as FC=2^{-ΔΔCT}=2^{-( Δ̅C̅T̅}^{_{group1}}^{- Δ̅C̅T̅}^{_{group2}}). To test the reliability of the assay, the 23 miRNAs of interest were tested twice on 20 randomly selected samples. The mixed effect model based method was used to calculate the coefficient of variation (CV) and intra-class correlation (ICC) on the ΔCT values of each marker (27). Only samples with detected values were used in the calculation. An average CV of 2.44% (range: 0,016%42.6%) was observed across the markers, indicating good reproducibility of the assay. The ICC ranges from 8% to 99.9%, with a median value of 83%. ICC measures the percentage of variation contributing to the variation among individuals. This value is high when the variance component associated with individual samples greatly exceeds the variation of the assay. ICCs of 15 of the markers exceed 70% in our data.

For the discovery cohort we used n=20 samples. We estimated that the mean for the coefficients of variation (CV) of the assay to be 2.44%. The calculation is based on the method described by Gail et al (27). By the delta method, we could approximate the variance of the ln(ΔCT) by the CV of the measurement. The effect size (ES) is defined as

$$E S = \frac{{\overline{ln ( Δ T )}}_{group 1} - {\overline{ln ( Δ T )}}_{group 2}}{std ( Δ T )} = \frac{ln ( FC )}{\sqrt{CV}} ,$$

thus, the fold change can be calculated as exp(ES×\sqrt{CV}).
For the validation cohort, we used a total of n=88 cases. We applied the Bonferroni adjustment, with a p-value cut off of 0.05/23-0.002 to control the overall family wise error rate (FWER) at 0.05. A total of 23 markers passed the initial screening and were formally tested using all 88 samples. With 20 samples from the healthy individuals and at least 14 samples in the disease group, there is at least 90% power to detect an effect size of 1.72 at the log level with a two-sided Wilcoxon rank-sum test (a=0.05/23=0.002). This translates into a FC of 1.3.
Hierarchical clustering analysis was applied to the ΔCT values. Markers that were consistently detected across all groups in the expansion cohort (missing data rate <30%) were used in the clustering analysis.

Wilcoxon rank-sum tests were used to search for differentially expressed miRNAs between two groups. Only markers that were consistently detected in both groups (total number of samples with non-missing values >5) were used in the differential gene analysis. The Benjamini and Hochberg's method was used to control the false discovery rate (FDR) at 20% (28). Principle Component Analysis (PCA) was applied to the differentially expressed miRNAs to reduce the dimension and to visualize the clusters. Linear Discriminant Analysis (LDA) was used to generate a three-marker model to classify samples of different groups (29, 30). Area Under the Receiver Operating Characteristic (AUROC) curve, sensitivity and specificity were calculated for each model. We used leaved-one-out cross validation (LOOCV) to avoid overfitting of the data.

### 6.2 Results

Reproducibility and Reliability of Our Approach.

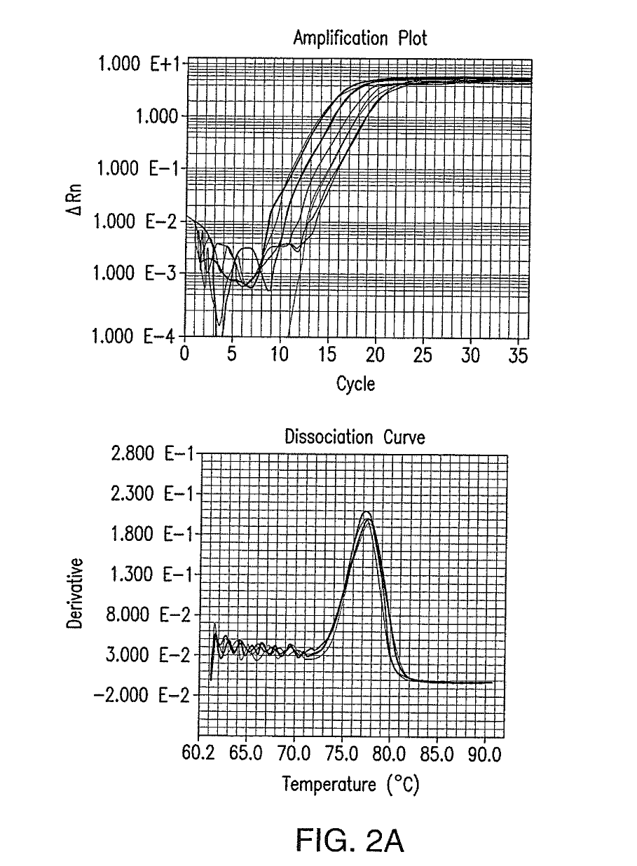

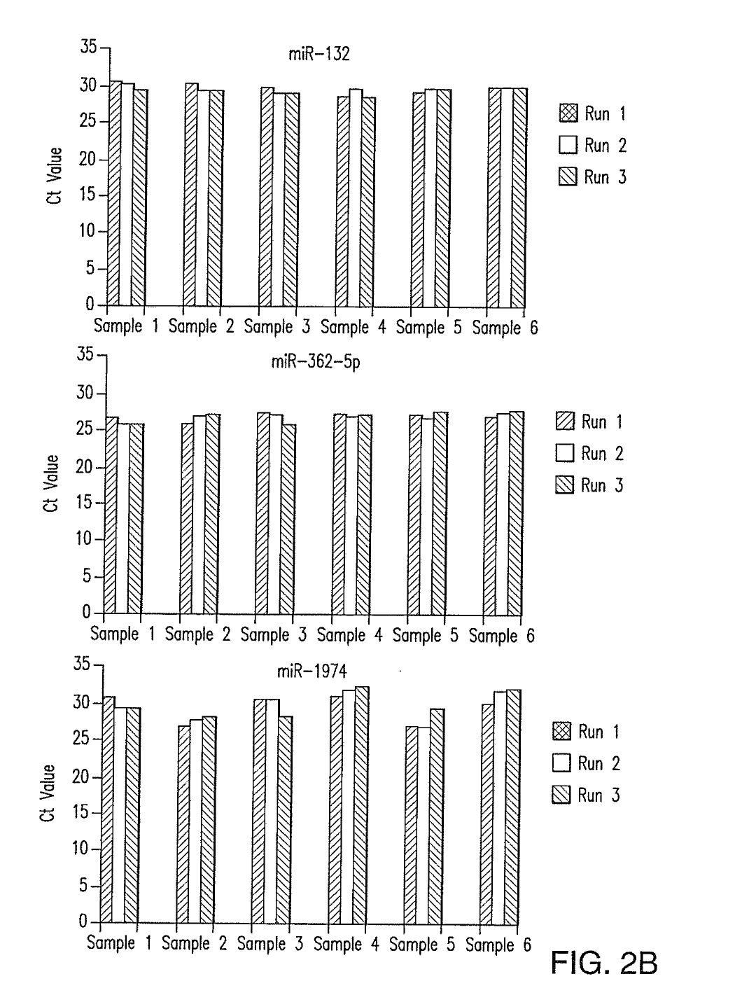

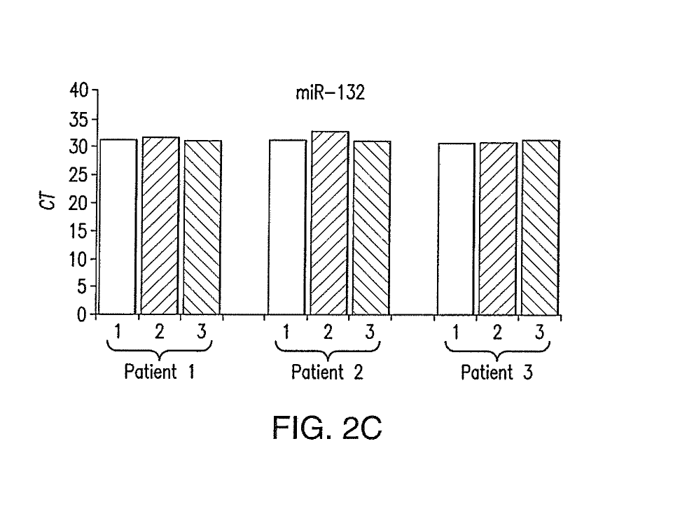

Our overall experimental design for miRNA profiling is outlined in FIG. 1. First, we confirmed that our extracted plasma miRNAs accurately reflect the original plasma miRNA population by adding spike-ins of serially diluted, synthetic miR-210, into aliquots of a randomly selected plasma sample from a healthy subject. Following miRNA extraction, we consistently detected miR-210 in a linear fashion by RT-qPCR, validating our miRNA extraction method (FIG. 2A). Next, we examined the reproducibility of our RT-qPCR protocol by two approaches. First, expression of three randomly selected miRNAs, miR-132, 362-5p, and 1974 was measured in three independent RT-qPCR assays using miRNAs extracted from six plasma samples. The CT values of all three runs in each plasma sample for each of the three miRNAs were highly consistent among measurements (FIG. 2B), demonstrating that extracted plasma miRNAs can be reproducibly detected by our RT-qPCR approach. Second, we examined miR-132 expression in three consecutive plasma samples collected one month apart from each of three preoperative EAOC patients. Remarkably, consistent expression of miR-132 is detected in all three samples from each of these patients (FIG. 2C).

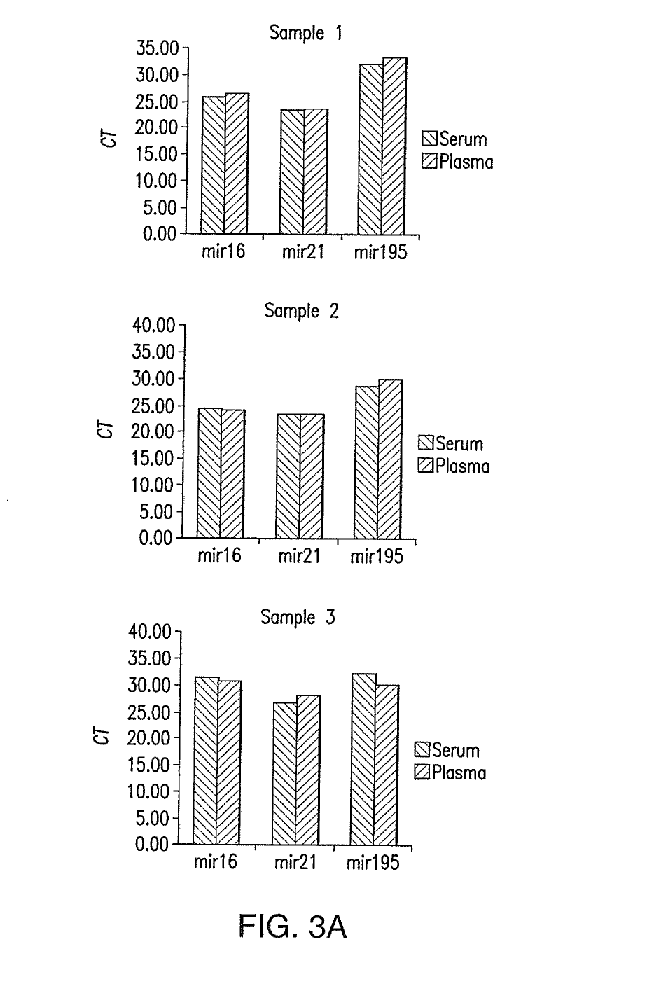

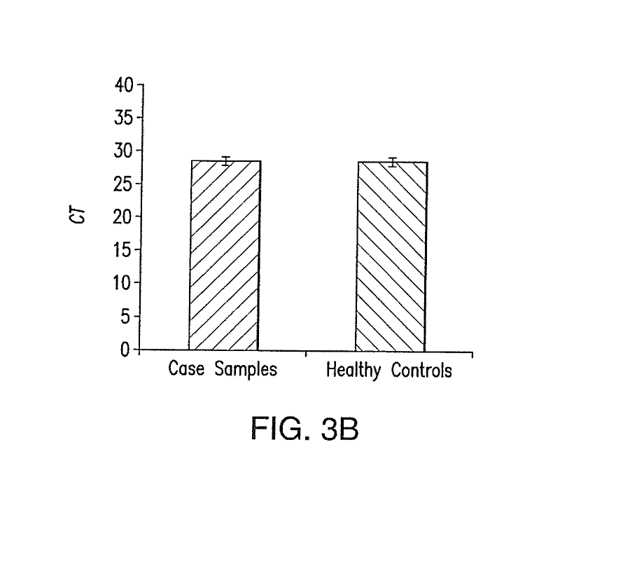

Because the peripheral blood used in this study was drawn in heparinized tubes and heparin is a known inhibitor of RT-PCR reaction, the robustness of our RT-qPCR was tested by comparing the expression of miR-16, 21, and 195 in three plasma samples collected in heparinized tubes and in matched serum samples collected in tubes without anticoagulant from EAOC patients. Comparable expression levels of the three miRNAs between plasma and serum samples suggest that the influence of heparin on our RT-qPCR is minimal (FIG. 3A). Despite similar blood collection protocols, our case and control plasma samples are from different institutions. Thus, we examined whether different sources of plasma samples may affect miRNA measurements in our study. Clearly, no difference was detected when miR-132 expression was examined by RT-qPCR between purchased healthy controls and endometriosis and EAOC samples collected at our institution (FIG. 3B).

Global miRNA Profiling in Patient Plasma.

The clinical demographics of all cases used in this study are summarized in Table 2. The majority of patients in the EAOC, endometriosis, and SOC categories were Caucasian (100%, 88%, and 100%, respectively) whereas the control group contained individuals more evenly distributed among the Caucasian, African-American, and Hispanic categories (45%, 35%, and 20%, respectively, p<0.0001). As expected, the endometriosis patients were younger than those with cancer and had lower gravidity and parity scores. Sixty-four percent of the EAOC cases were stage I or II whereas only 14% of the SOC cases were early stages, consistent with the more aggressive phenotype and late diagnosis seen in patients with SOC (31). There were no significant associations with body mass index, tobacco or alcohol use. Of the EAOC cases (n=14), 43% (n=6) were clear cell and 50% (n=7) were endometrioid tumors, while 7% (n=1) was with mixed clear cell/endometrioid histology. Presence of concurrent endometriosis at the time of cancer diagnosis was confirmed by pathology in 36% EAOC cases (n=5), of which two were endometrioid, two were clear cell, and one was mixed endometrioid/clear cell tumor.

Since the genome-wide circulating miRNA expression profile in endometriosis and EAOC has not been established to date, we performed global plasma miRNA expression profiling by RT-qPCR in the discovery phase of our study. Because the purpose of our study is to identify plasma miRNAs that can serve as biomarkers to distinguish endometriosis and EAOC samples from healthy controls, expression of 1113 human miRNAs was measured in 20 plasma samples from healthy individuals (n=6), endometriosis patients (n=7), and EAOC patients (n=7) to pick candidate miRNAs that are expressed in these sample categories and have potential to differentiate different groups of diseases. Of the 1113 miRNAs, miRNAs that were not expressed in any of the samples were eliminated first. Of the remaining 286 miRNAs, miRNAs that were not expressed in at least four samples in any sample category were further removed. Finally, 23 miRNAs with a minimal differential average CT value of two (ΔCT≧2) among EAOC, endometriosis, and healthy controls, and miR-132, a potential endogenous control miRNA, were selected as candidate biomarkers for further study (Table 3). Expression of these 24 miRNAs was confirmed in independent RT-qPCR runs using the 20 samples in the discovery cohort.

Unique Plasma miRNA Signatures can Differentiate Between Patient Categories.

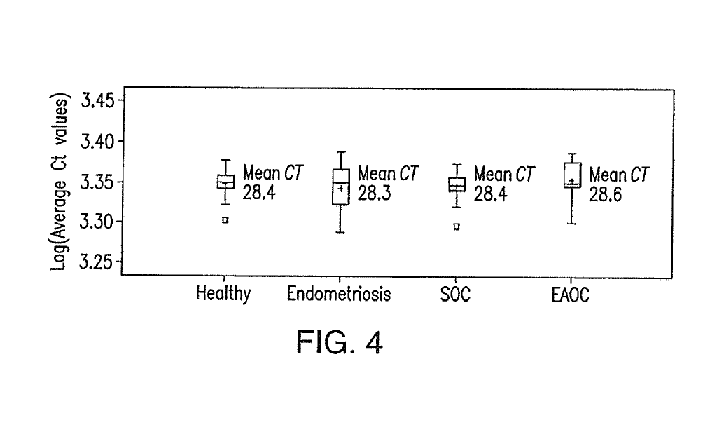

The 24 candidate miRNAs were next validated by RT-qPCR in a total of 67 samples (healthy, n=20; endometriosis, n=33; EAOC, n=14), including the 20 samples from the discovery phase. The serous ovarian cancer (SOC) cases (n=21) were added as non-endometriosis-associated controls (15). Currently, there is no consensus on housekeeping miRNAs used for plasma miRNA RT-qPCR data normalization. In our study, we found that miR-132 is the most consistently expressed miRNA across all samples from all categories (FIG. 4). Thus, miR-132 was subsequently employed as an endogenous control for plasma miRNA RT-qPCR data normalization.

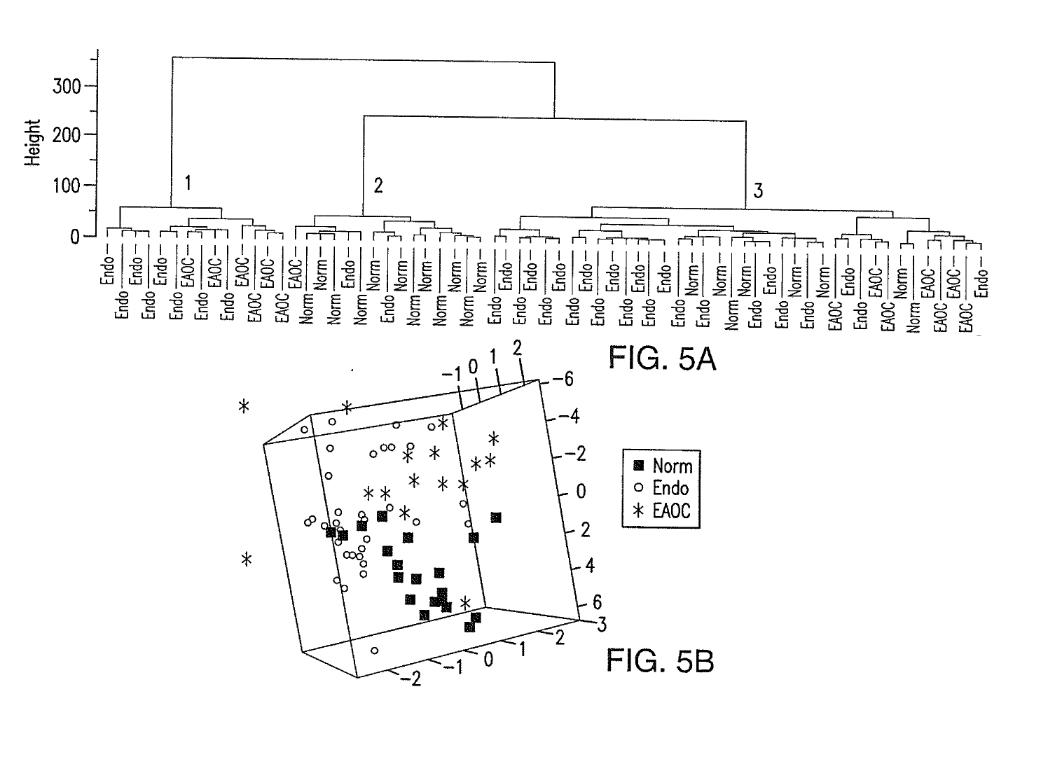

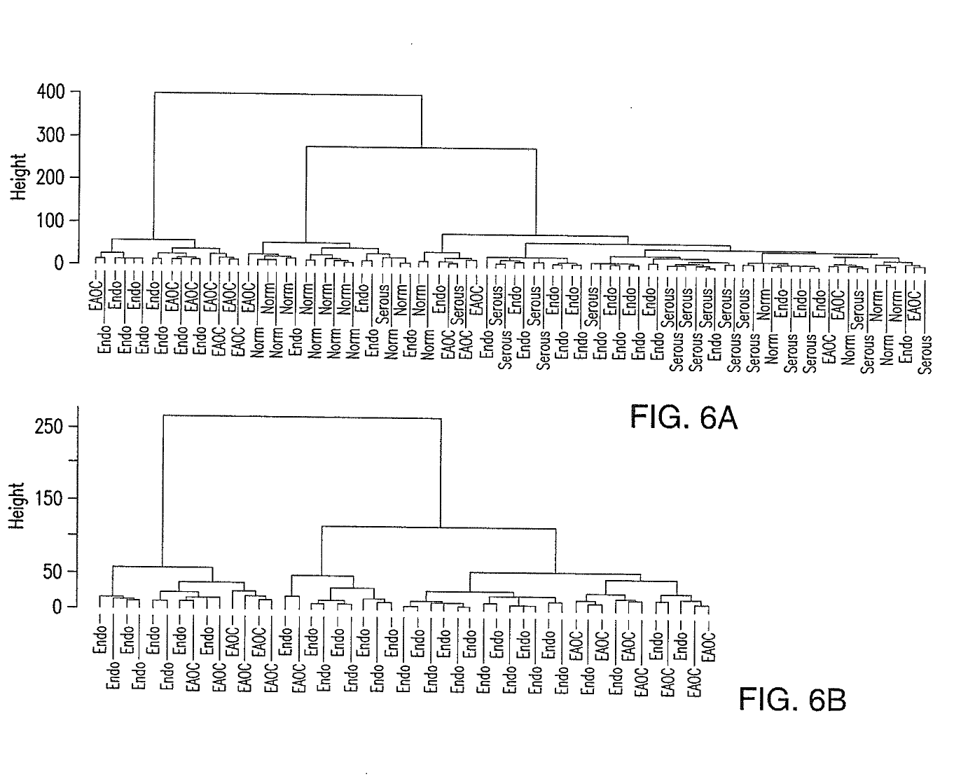

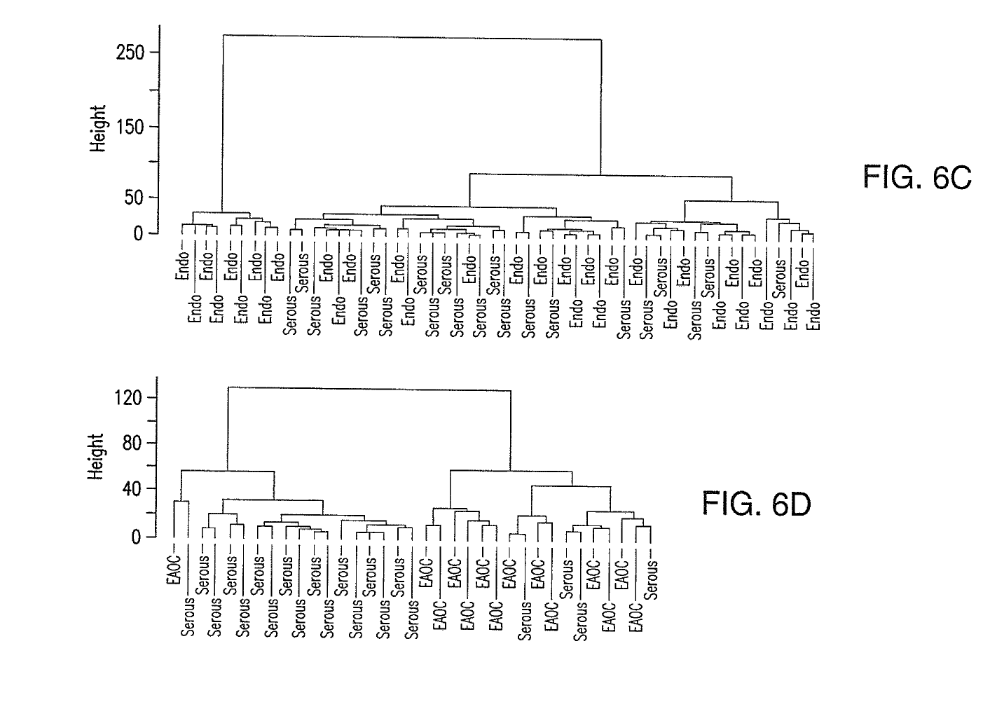

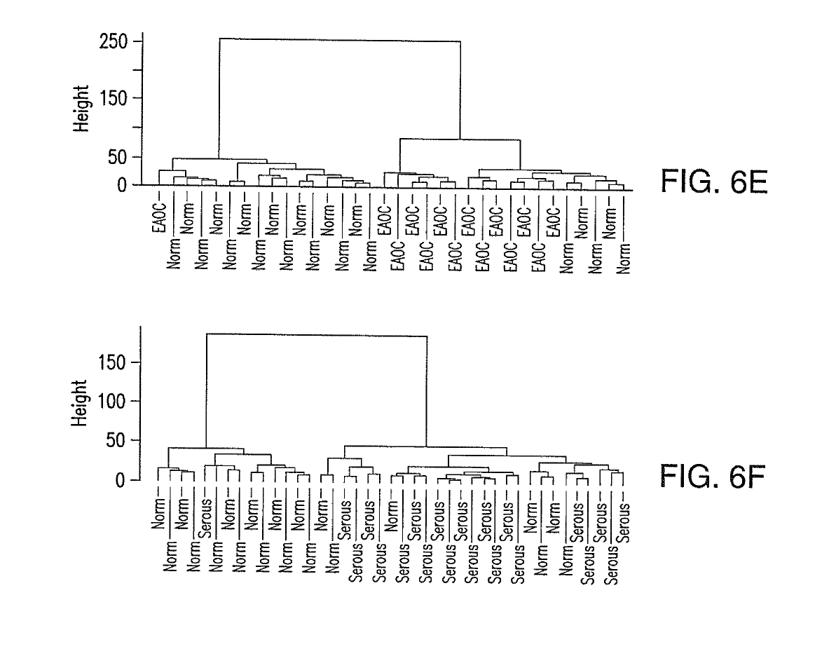

The unsupervised clustering analysis using the expression of the candidate plasma miRNAs in healthy controls (n=21), endometriosis (n=33), and EAOC (n=14) shows three distinct clusters: cluster 1 is enriched with endometriosis (n=8) and EAOC (n=6) samples; cluster 2 is enriched with normal samples with two endometriosis cases and one EAOC case misclassified in this cluster; cluster 3 has two sub-clusters. One is enriched with endometriosis samples with six healthy controls misclassified in this sub-cluster; the other is enriched with EAOC cases (n=7) but with misclassified endometriosis (n=3) and healthy controls (n=2) (FIG. 5A). We also used principle component analysis (PCA) to aid the visualization of the data at lower dimension. Consistent with our clustering analysis, the plot of the first three principle components of the data also shows that the three groups form three distinct but not completely separated clouds (FIG. 5B). These results reveal the potential of using plasma miRNA expression patterns for classification of these diseases. When the unsupervised clustering analysis was performed in all cases (n=88, including SOC), the 21 SOC cases were found to be interspersed into the other three categories and cannot be separated from other categories of diseases (FIG. 6A). Similarly, an unsupervised clustering in pair-wise comparison of endometriosis/EAOC or endometriosis/SOC (FIGS. 6B, 6C) showed that while the majority of endometriosis and EAOC samples can be classified into relatively distinct clusters, the SOC samples are mixed with endometriosis samples. However, when EAOC and SOC samples were compared, the 23-miRNA signature correctly classified the majority of them into two major clusters (93% and 81%, respectively, FIG. 6D), and when unsupervised clustering was performed between healthy controls and SOC or healthy controls and EAOC, the 23-miRNA signature can also correctly classify cancer samples from controls (FIGS. 6E, 6F). Overall, these results suggest that although the 23-miRNA signature is reflective of its originating clinical entity (endometriosis and EAOC from the discovery cohort), it also contains commonly dysregulated plasma miRNAs across different EOC histotypes. Thus, optimization of this list of candidate miRNAs may provide novel plasma biomarkers for detection and classification of endometriosis and EAOC.

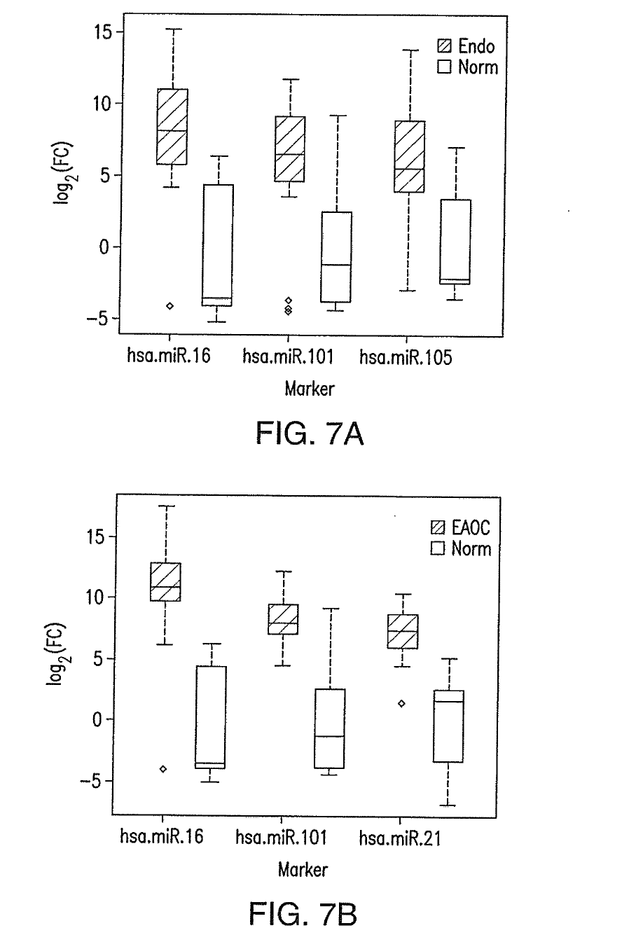

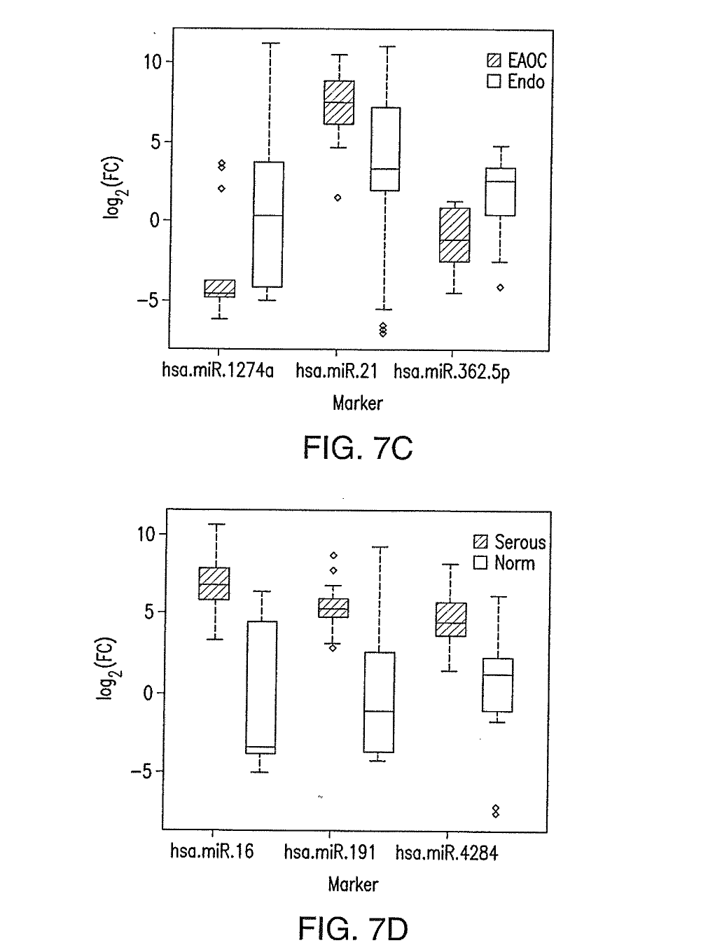

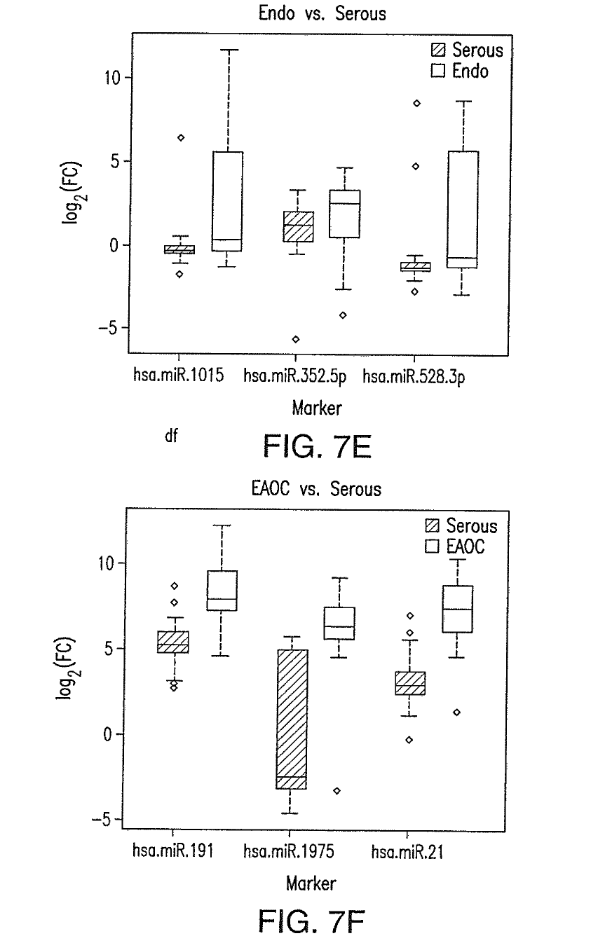

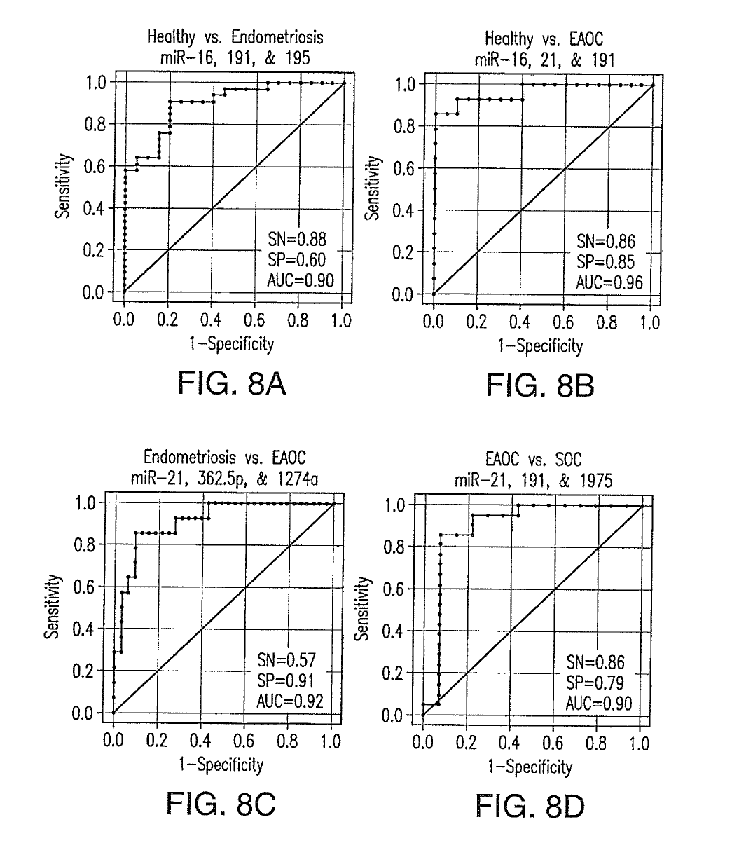

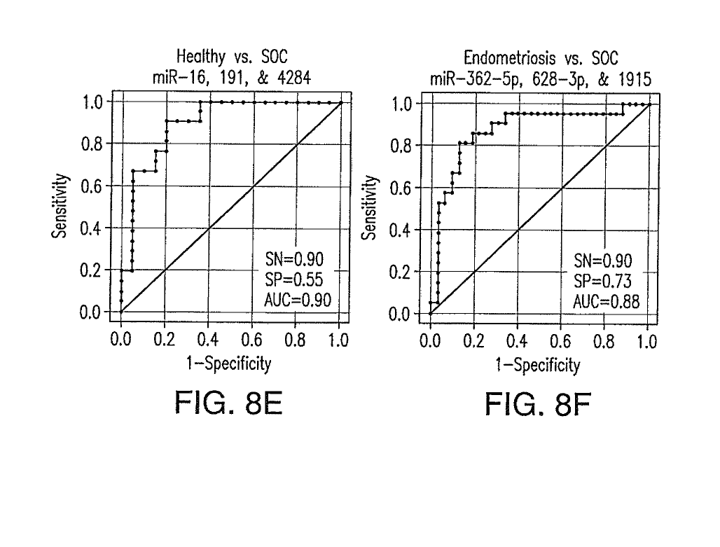

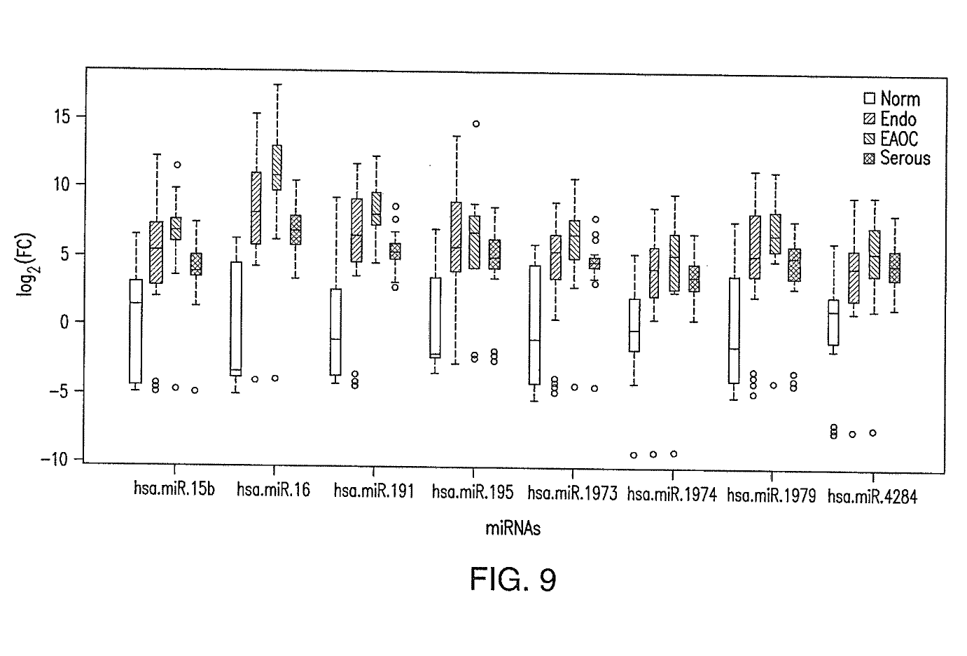

We then compared the expression of the 23 miRNAs between all categories (healthy controls, n=20; endometriosis, n=33; EAOC, n=14; and SOC, n=21) in the validation cohort to identify the top 10 differentially expressed miRNAs between any two categories by Wilcoxon Rank Sum test (Tables 4 and 5). The top three candidate miRNAs for each of the pair-wise comparisons are shown in FIG. 7. While the majority of the top differentially expressed plasma miRNAs in both EAOC and SOC samples are overexpressed compared to healthy controls, the level of expression in EAOC samples is generally much higher than in SOC samples (FIG. 7F). Next, we examined which combination of miRNAs among the candidate miRNAs could differentiate between the sample groups with the highest predictive power by linear discriminant analysis (LDA). Leave-one-out cross validation (LOOCV) was used to avoid overfitting of the data. Application of LDA revealed three plasma miRNAs, miR-16, 191, and 195, all upregulated in endometriosis, that could differentiate between healthy and endometriosis cases with 88% sensitivity (SN), 60% specificity (SP) (FIG. 8A). A combination of miR-16, 21, and 191 can differentiate between healthy and EAOC with 86% SN and 85% SP (FIG. 8B), while miR-21, 362-5p, and 1274a can differentiate between endometriosis and EAOC with 57% SN and 91% SP (FIG. 8C). miR-21, 191, and 1975 together could distinguish between EAOC and SOC with 86% SN and 79% SP (FIG. 8D). Expression signature of miR-16, 191, and 4284 could be used for discerning healthy individuals from SOC patients with 90% SN and 55% SP (FIG. 8E), while miR-362-5p, 628-3p, and 1915 can differentiate endometriosis and SOC with 90% SN and 73% SP (FIG. 8F). Interestingly, we also noticed a general trend of elevated plasma miRNA expression from healthy controls to endometriosis to EAOC but not in SOC samples (FIG. 9), suggesting that these miRNAs may serve as novel biomarkers that reflect the pathological progression from benign to precursor lesion to fully developed EAOC. Altogether, we have identified different panels of plasma miRNAs that may serve as novel biomarkers to discriminate between healthy, endometriosis, EAOC, and SOC patients.

Validation of Human miRNA Signature in a Preclinical Mouse Model.

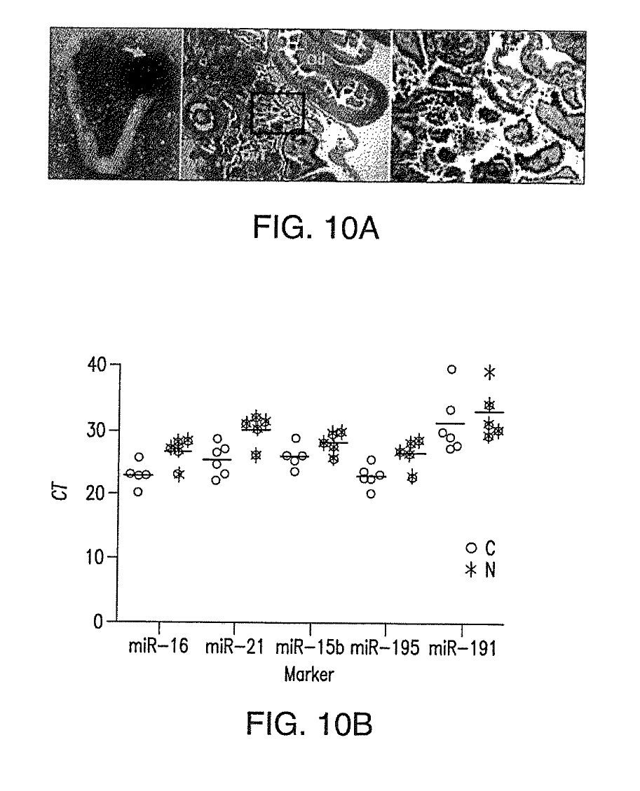

Mouse models of human diseases are powerful tools to study the mechanism of disease and test the efficacy of pre-clinical therapeutics. We next tested whether some of the most differentially expressed miRNAs between healthy controls and EAOC patients (Table 4) are also differentially expressed between healthy mice and mice with endometrioid ovarian tumors. To achieve this, we employed a previously described, conditional mouse model for endometriosis-associated endometrioid ovarian cancer (25). The LSL-Kras^{G12D/+}/Pten^{loxp/loxp} conditional mice develop orthotopic tumors 12 weeks post AdCre injection under the ovarian bursa (FIG. 10A) (25). We induced tumors in six female mice, sacrificed the mice when moribund, and collected serum at necropsy. Five healthy (non-injected), age-matched female mice were sacrificed as controls. Serum miRNAs were extracted and subjected to RT-qPCR analysis to measure expression of miR-15b, 16, 21, 191, and 195. Among the top 10 differentially expressed human miRNAs between healthy controls and EAOC patients (Table 4), these five are the only miRNAs that have mouse orthologs. Our results demonstrate that four of the five miRNAs can also delineate the EAOC mice from healthy controls (FIG. 10B), suggesting a potential EAOC-specific pathogenesis leading to the dysregulation of miR-15b, 16, 21, and 195, and further validating the biological relevance of the plasma miRNA expression signature we have identified as biomarkers of human EAOC.

Ovarian Tumor Tissue and Corresponding Plasma have Distinct miRNA Expression Profiles.

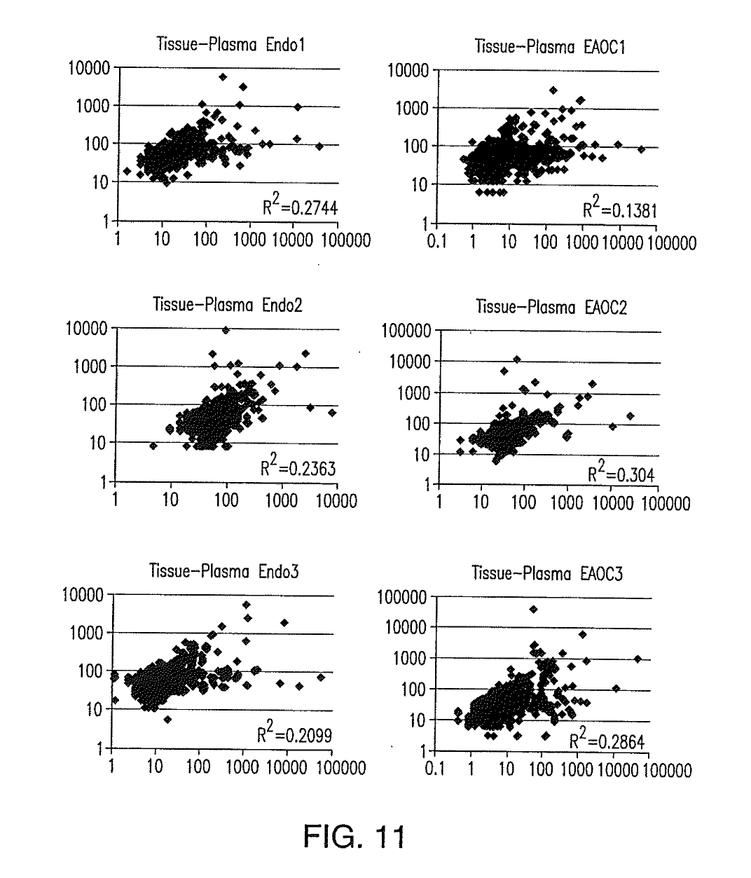

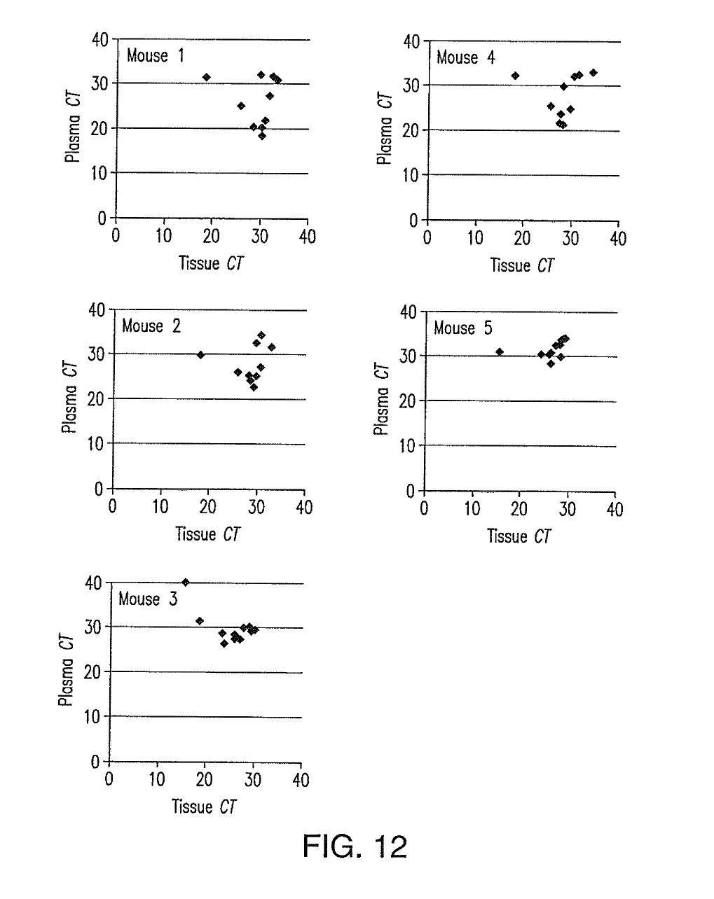

Numerous miRNAs have been reported to be dysregulated in ovarian tumors (8, 32). Despite the great potential circulating miRNAs hold as novel biomarkers for classification and early diagnosis of EOC, it remains unclear whether miRNAs in patient plasma reflect miRNA expression occurring in corresponding diseased tissues. To address this question, we profiled miRNA expression in six pairs of endometriosis tissue or EAOC primary tumors and corresponding plasma samples using the NanoString technology (33), which provides digital counting of miRNA copy numbers without the need for miRNA amplification. While we detected a very modest correlation of overall miRNA expression in paired tissue and plasma samples, we also observed distinct miRNA expression profiles, especially among the highly expressed miRNAs (FIG. 11). Specifically, while miR-16, 21, and 132 were consistently ranked as the top three most highly expressed plasma miRNAs, only miR-21 was consistently ranked among the top five most highly expressed tissue miRNAs. To further support our conclusion, we examined the expression of 10 miRNAs with mouse orthologs (miR-15b, 16, 21, 132, 191, 195, 362-5p, 652, 744, and 1274a) from the 24 miRNAs identified from the discovery cohort (Table 3) in five paired tumor and plasma samples from tumor-bearing LSL-Kras^{G12D/+}/Pten^{loxp/loxp} mice. No significant correlation was detected in the paired samples from all five mice (FIG. 12), mirroring the findings in humans. Our data suggest that plasma and tissue samples have distinct miRNA expression profiles, Thus, differentially expressed miRNAs identified through comparing normal and tumor tissues as reported in numerous studies cannot be simply applied to study plasma/serum samples.

### 6.3 Discussion

In this study, we performed global profiling of circulating miRNAs in endometriosis and EAOC samples. We have identified unique plasma miRNA expression signatures that distinguish endometriosis and EAOC patients from healthy controls, suggesting that circulating miRNAs may serve as promising biomarkers with high sensitivity and specificity for early detection and diagnosis of endometriosis and EAOC. Remarkably, four out of five miRNAs (miR-15b, 16, 21, and 195) we found differentially expressed in human EAOC from healthy controls show a similar expression pattern in a preclinical mouse model for EAOC, providing a strong support for the validity of our results and also suggesting that changes in these miRNA levels are likely due to disease-specific pathogenesis.

In addition to causing pain and infertility, endometriosis is also considered a precursor of EAOC, as supported by a growing number of epidemiological and molecular studies (15, 34). Frequent mutations of a tumor suppressor gene, ARID1A, have not only been identified in clear cell and endometrioid ovarian tumors, but have also been found in concurrent endometriosis and atypical endometriosis lesions (34), suggesting loss of ARID1A function to be an early step in the transformation of endometriosis to EAOC (34). Although the complete pathway remains unclear, these notable findings strongly support the molecular links between endometriosis and EAOC (35), and pave the way for developing new, reliable biomarkers that can not only aid in the diagnosis of endometriosis and EAOC, but also identify endometriosis patients at risk for developing EAOC. The plasma miRNA signatures reported in our study, focused on distinguishing endometriosis or EAOC from healthy controls, is particularly valuable since these biomarkers may potentially be utilized in a well-defined high-risk population, such as patients with prolonged history of suspected or confirmed endometriosis and with other ovarian cancer predisposing risk factors like age, reproductive history, or family history of ovarian cancer.

It is estimated that a screening test for ovarian cancer would require a sensitivity of at least 75% and a specificity of more than 99.6% to achieve a positive predictive value (PPV) of 10%, the minimum PPV required for a screening test. Despite extensive efforts to develop new protein biomarkers for early detection of ovarian cancer, CA125 still stands to be the most dependable of all biomarkers examined to date (36). However, even CA125 falls short of the requirement for sensitivity and specificity to be useful as a biomarker for ovarian cancer screening (36, 37). Ovarian cancer is a highly heterogeneous disease and the four main histological subtypes of ovarian cancer are now considered different diseases, which may develop differently, respond differently to chemotherapy, and express different sets of biomarkers (38). However, in the majority of past biomarker development studies, ovarian cancer has been largely regarded as a single entity. This may at least partially account for the failed effort to develop biomarkers for early detection of ovarian cancer. Our results further support this concept by demonstrating that EAOC and SOC are different clinical entities and can be distinguished based on plasma miRNA expression profiles (FIG. 6D). Although the sensitivity and specificity values of the plasma miRNA signatures reported here are lower than required to be applied in clinical practice yet, our study nevertheless serves as a foundation for future follow-up studies with larger sample sizes. In addition, combining histotype-specific plasma miRNA expression signatures with CA125 may be a promising strategy to improve the sensitivity and specificity of ovarian cancer early detection.

The panel of plasma miRNAs we identified clearly demonstrates that circulating miRNAs are promising novel biomarkers for early detection of EAOC. Surprisingly, when we compared the global miRNA expression in primary EAOC tumors or endometriosis tissues to corresponding plasma samples, only a very modest correlation was observed (FIG. 11). Interestingly, neovascularization is important for pathogenesis of endometriosis and EAOC and the lesions of endometriosis and EAOC are highly vascular (39, 40). Despite no consensus on the cellular origin of circulating miRNAs at present (41), the lack of correlation between paired tissue-plasma miRNA expression profiles in both endometriosis and EAOC patients and in EAOC mouse model (FIGS. 11 and 12) strongly suggests that disease tissue or malignant tumor cells are not the sole source of circulating miRNAs. Since loco-regional inflammation plays an important role in endometriosis and EAOC pathogenesis (42, 43), it is more likely that the miRNA signatures we detected in patient plasma actually reflect the output of a systematic response of host microenvironment to the disease. Currently, the majority of circulating miRNA biomarker studies is based on primary tumor miRNA expression profiles. In light of our results, by not performing independent global miRNA profiling in plasma/serum samples, these studies may have missed many relevant biomarker candidates.

Numerous miRNAs have been reported to be dysregulated in EOC (8, 44), among which miR-21 and members of the miR-200 family are the most consistently upregulated compared to normal controls. Because SOC accounts for a majority of EOC cases, few reports have focused on identifying miRNA expression signatures in other EOC histotypes. Upregulation of miR-21, miR-203, and miR-205 were found to be specific to the endometrioid histotype and miR-222 was downregulated in EAOC samples (8). By using next generation sequencing, a different set of miRNAs that are specifically upregulated in endometrioid and clear cell histotypes have also been reported recently (44), of which miR-9, 96, 182, 183, 196a, 196b, 205, and 375 are specifically upregulated in endometrioid histotype, and miR-30a, 30a*, and 486-5p are upregulated in clear cell histotype. Among the top 10 plasma miRNAs that are most differentially expressed between EAOC and healthy controls (Table 4), miR-21 is the only miRNA that overlaps with the EAOC-specific miRNA signature derived from tumor tissues (8). This discrepancy further supports our conclusion that tumor cells are not a major source of circulating miRNAs. Interestingly, miR-21 has been reported as one of the most consistently overexpressed oncomiRs in almost all tumor types (45), raising the possibility that the highly elevated miR-21 expression in the plasma of EAOC patients may reflect activation of a common oncogenic pathway that contributes to EAOC pathogenesis, despite the source of circulating miR-21 remains unknown.

Circulating miRNAs hold great promise as biomarkers on cancer early detection, diagnosis, and prognosis. Plasma/serum miRNA signatures have been reported in almost all tumor types, such as in lung (13, 46), gastric (47), breast (48), pancreatic (14), and ovarian cancers (49, 50). Among the three miRNAs that comprise the signature that distinguishes EAOC from healthy controls (FIG. 8B), overexpression of plasma/serum miR-16 and miR-21 has been reported in many tumor types (14, 46, 47), including in ovarian cancer (49, 50). However, dysregulated circulating miR-191 expression has not been implicated in any cancers to date. Thus, the combination of miR-16, 21, and 191 may represent a unique signature to EAOC.

Despite our rigorous statistical methods for signature identification, we acknowledge the limited sample size of our studies and the need for further validation of findings in larger cohorts. In addition, inclusion of cases with atypical endometriosis, concurrent endometriosis-EAOC cases, and of early stage EAOC is warranted in future studies to further validate our miRNA signatures.

TABLE 2
Demographic and clinical characteristics of the study cohort.

| | | Normal | Endo | SOC | EAOC^{a} | |
|---|---|---|---|---|---|---|
| | ALL | n = 20 | n = 33 | n = 21 | n = 14 | |
| Characteristic | n = 88 | (22.73%) | (37.50%) | (23.86%) | (15.91%) | p-value^{b} |
| Age at | 46.84(17.41) | 38.75(14.11) | 36.24(10.20) | 65.52(12.46) | 55.3 [symbol] (14.61) | <0.0001 |
| presentation | | | | | | |
| (year), mean(SD) | | | | | | |
| Gravidity, median | 1(0.3) | NA^{d} | 0(0.1) | 2.5(1.3) | 0.5(0.2) | 0.0092 |
| (IQR)^{b} | | | | | | |
| Parity, median | 1(0.2) | NA | 0(0.1) | 2.5(1.3) | 0.5(0.2) | 0.0071 |
| (IQR)^{c} | | | | | | |
| Body mass index | 26.27(5.36) | NA | 26.27(5.09) | 24.94(5.43) | 27.92(5.85) | 0.4417 |
| (kg/m2), | | | | | | |
| mean(SD) | | | | | | |
| Race (White), n | 73(83.91) | 9(45.00) | 30(90.91) | 20(100.00) | 14(100.00) | <0.0001 |
| (%) | | | | | | |
| History of | 19(29.23) | NA | 9(29.03) | 7(35.00) | 3(21.43) | 0.7240 |
| tobacco use, n | | | | | | |
| (%) | | | | | | |
| History of alcohol | 31(48.44) | NA | 17(54.84) | 7(35.00) | 7(53.85) | 0.6835 |
| use, n (%) | | | | | | |
| History of | 19(65.22) | NA | 17(68.00) | 0(0) | 2(66.67) | 0.7186 |
| endometriosis, n | | | | | | |
| (%) | | | | | | |
| History of oral | 26(81.25) | NA | 21(84.00) | 2(66.67) | 3(75.00) | 0.5445 |
| contraceptive pill | | | | | | |
| use, n (%) | | | | | | |
| Postmenopausal. | 21(34.43) | NA | 2(6.45) | 12(70.59) | 7(53.85) | 0.0002 |
| n(%) | | | | | | |
| Family history of | 22(45.16) | NA | 11(37.93) | 11(55.00) | 6(46.15) | 0.4710 |
| cancer, n (%) | | | | | | |
| Stage of disease, | | NA | NA | | | |
| n(%) | | | | | | |
| Early | 11(31.43) | | | 3(14.29) | 8(57.14) | 0.0084 |
| Late stage | 24(68.57) | | | 18(85.71) | 6(42.86) | |

^{a}EAOC group comprises n = 6 endometrioid (50%). n = 7 cloar cell tumors (48.2%) and n = 1 (7.1%) mixed histology of clear cell/endometrioid type
^{b}ANOVA or Kruskal-Wallis test for continuous variables; Chi-square test or Cochran-Mantel-Haenszel Chi-square test for categorical variables
^{c}IQR. Interquartile Range (25^{th} percentile, 75^{th} percentile)
^{d}NA, not available [symbol]
indicates data missing or illegible when filed

TABLE 3
List of the 24 miRNAs identified by genome-wide plasma miRNA
expression profiling from healthy controls (n = 6), endometriosis patients
(n = 7) and EAOC patients (n = 7).

| hsa-miR- | hsa-miR- | hsa-miR- | hsa-miR- | hsa-miR- | hsa-miR- | hsa-miR- | hsa-miR- |
|---|---|---|---|---|---|---|---|
| 1978 | 1974 | 1977 | 16 | 1973 | 195 | 191 | 652 |
| hsa-miR- | hsa-miR- | hsa-miR- | hsa-miR- | hsa-miR- | hsa-miR- | hsa-miR- | hsa-miR- |
| 1308 | 1975 | 15b | 1979 | 21 | 766 | 628-3p | 4313 |
| hsa-miR- | hsa-miR- | hsa-miR- | hsa-miR- | hsa-miR- | hsa-miR- | hsa-miR- | hsa-miR- |
| 1246 | 376a | 744 | 1274a | 1915 | 362-5p | 4284 | 132 |

TABLE 4
The 10 most differentially expressed miRNAs in pair-wise
comparisons among healthy controls, endometriosis and EAOC samples.

| | W. pvalue¹ | a. W. pvalue² | FC³ |
|---|---|---|---|
| Endomotriosis (n = 33) vs. | | | |
| Healthy Controls (n = 20) | | | |
| hsa-miR-16 | 0.00000 | 0.00001 | 396.62 |
| hsa-miR-195 | 0.00001 | 0.00011 | 62.02 |
| hsa-miR-191 | 0.00004 | 0.00028 | 79.05 |
| hsa-miR-1974 | 0.00007 | 0.00034 | 15.43 |
| hsa-miR-4284 | 0.00025 | 0.00101 | 16.79 |
| hsa-miR-15b | 0.00041 | 0.00124 | 26.27 |
| hsa-miR-1978 | 0.00041 | 0.00124 | 51.29 |
| hsa-miR-1979 | 0.00051 | 0.00135 | 30.54 |
| hsa-miR-362-5p | 0.00062 | 0.00149 | 3.41 |
| hsa-miR-1973 | 0.00113 | 0.00259 | 16.69 |
| EAOC (n = 14) vs. Healthy | | | |
| Control (n = 20) | | | |
| hsa-miR-21 | 0.00001 | 0.00014 | 147.40 |
| hsa-miR-191 | 0.00001 | 0.00014 | 380.01 |
| hsa-miR-16 | 0.00002 | 0.00016 | 1323.18 |
| hsa-miR-15b | 0.00005 | 0.00032 | 80.50 |
| hsa-miR-1977 | 0.00029 | 0.00117 | 11.43 |
| hsa-miR-1979 | 0.00029 | 0.00117 | 99.11 |
| hsa-miR-1973 | 0.00038 | 0.00131 | 58.96 |
| hsa-miR-1974 | 0.00057 | 0.00170 | 20.75 |
| hsa-miR-4284 | 0.00073 | 0.00196 | 26.58 |
| hsa-miR-195 | 0.00137 | 0.00273 | 48.02 |
| EAOC (n = 14) vs. | | | |
| Endomotriosis (n = 33) | | | |
| hsa-miR-362-5p | 0.00014 | 0.00343 | 0.14 |
| hsa-miR-1274a | 0.01323 | 0.09634 | 0.13 |
| hsa-miR-21 | 0.01606 | 0.09634 | 13.84 |
| hsa-miR-766 | 0.02195 | 0.10534 | 15.11 |
| hsa-miR-1975 | 0.03142 | 0.11204 | 7.75 |
| hsa-miR-1308 | 0.03527 | 0.11204 | 0.13 |
| hsa-miR-191 | 0.03735 | 0.11204 | 4.81 |
| hsa-miR-744 | 0.05209 | 0.13192 | 4.89 |
| hsa-miR-376a | 0.05497 | 0.13192 | 6.38 |
| hsa-miR-1246 | 0.06112 | 0.14058 | 1.36 |

¹Wilcoxon p value, ²adjusted Wilcoxon p value, ³Fold change

TABLE 5
The 10 most differentially expressed miRNAs in pair-wise
comparisons between SOC patients and healthy controls,
endometriosis, or EAOC samples.

| | W. pvalue | a. W. pvalue | FC |
|---|---|---|---|
| SOC (n = 21) vs | | | |
| Healthy (n = 20) | | | |
| hsa-miR-4284 | 0.00028 | 0.00504 | 6.96 |
| hsa-miR-1974 | 0.00086 | 0.00774 | 8.56 |
| hsa-miR-16 | 0.00243 | 0.01458 | 4.56 |
| hsa-miR-1977 | 0.00393 | 0.01769 | 2.49 |
| hsa-miR-1975 | 0.00728 | 0.02621 | 0.39 |
| hsa-miR-195 | 0.02323 | 0.06037 | 2.64 |
| hsa-miR-1978 | 0.02521 | 0.06037 | 2.59 |
| hsa-miR-21 | 0.02683 | 0.05037 | 2.08 |
| hsa-miR-362-5p | 0.03142 | 0.06284 | 1.76 |
| hsa-miR-15b | 0.04500 | 0.07747 | 2.21 |
| SOC (n = 21) vs | | | |
| Endo (n = 33) | | | |
| hsa-miR-15b | 0.00923 | 0.08226 | 0.23 |
| hsa-miR-191 | 0.01005 | 0.08226 | 0.27 |
| hsa-miR-1973 | 0.01743 | 0.08226 | 0.48 |
| hsa-miR-362-5p | 0.01828 | 0.08226 | 0.52 |
| hsa-miR-16 | 0.06761 | 0.22134 | 0.25 |
| hsa-miR-21 | 0.07378 | 0.22134 | 0.25 |
| hsa-miR-195 | 0.10272 | 0.26414 | 0.31 |
| hsa-miR-1308 | 0.23086 | 0.51146 | 0.49 |
| hsa-miR-1974 | 0.25573 | 0.51146 | 0.59 |
| hsa-miR-652 | 0.30405 | 0.54729 | 0.56 |
| SOC (n = 21) vs | | | |
| EAOC (n = 14) | | | |
| hsa-miR-21 | 0.00006 | 0.00102 | 0.06 |
| hsa-miR-16 | 0.00014 | 0.0011 | 0.05 |
| hsa-miR-191 | 0.00021 | 0.0011 | 0.12 |
| hsa-miR-15b | 0.00026 | 0.0011 | 0.13 |
| hsa-miR-1975 | 0.00126 | 0.00428 | 0.23 |
| hsa-miR-1246 | 0.00259 | 0.00734 | 0.33 |
| hsa-miR-362-5p | 0.00399 | 0.0088 | 3.71 |
| hsa-miR-1979 | 0.00414 | 0.0088 | 0.23 |
| hsa-miR-1973 | 0.01101 | 0.02011 | 0.29 |
| hsa-miR-195 | 0.01183 | 0.02011 | 0.24 |

### 7. EXAMPLE 2

ELISA.

CA-125 levels were determined in all plasma samples using the RayBio Human CA-125 ELISA kit (Cat#: ELH-CA125-001). Specimens, standards and reagents were prepared according to manufacturer's instructions. Plasma CA-125 concentrations were determined by measurement of absorbance at 450 nm, which was read against a standard curve. Levels were determined as units per milliliter (U/mL). The receiver operating characteristics (ROC) curves of CA125 were constructed for SOC, EAOC, endometriosis and healthy individuals.

CA-125 Levels can Differentiate Most SOC from Other Groups.

As a reference point for our miRNA measurements, we measured next plasma CA-125 (a standard tumor marker currently employed mostly for monitoring ovarian cancer response to therapy) in all patient categories (total n=80; SOC=21, Healthy=20, EAOC=15, endometriosis=24), by ELISA.

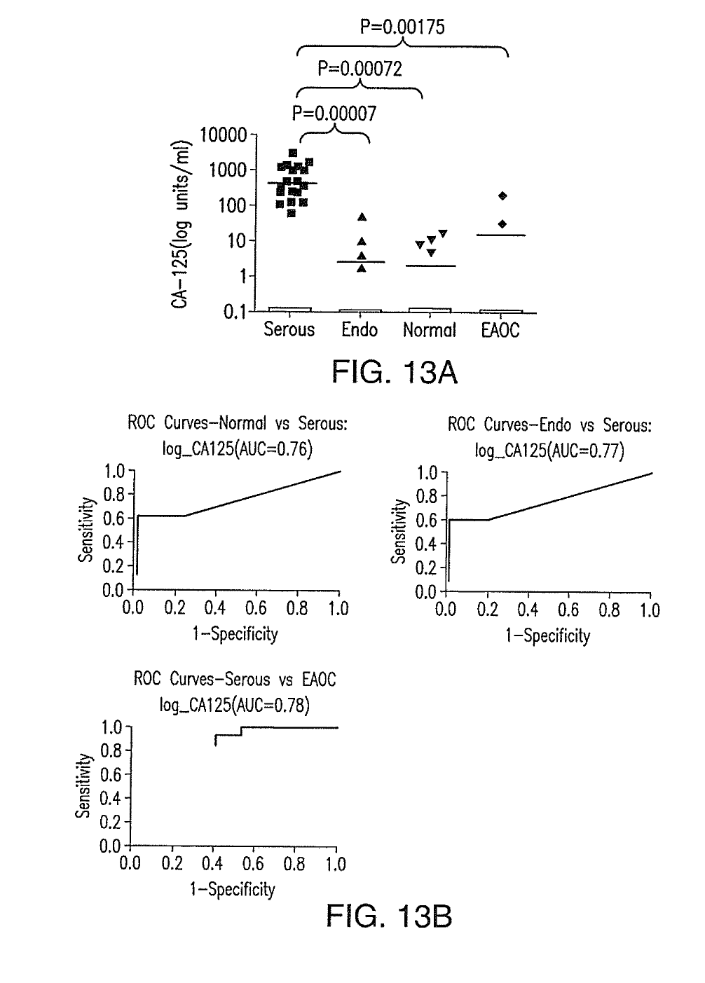

The results in FIG. 13A show that although most of the SOC plasma CA-125 levels were elevated above the normal limit (35 U/ml), there were, however, only two EAOC patients with high CA-125. Furthermore, one of the endometriosis cases also showed elevated CA-125. As expected, plasma from healthy cases had CA-125 lower than 35 U/ml (FIG. 13A). Wilcoxon analysis showed significant difference in CA-125 plasma levels in SOC and EAOC, endometriosis and healthy (P<0.05) (FIG. 13A). Generated ROC showed 76% AUC for comparison of healthy and SOC, 77% AUC for endometriosis and SOC and 76% AUC for EAOC and SOC (FIG. 13B). These results indicate that, in this study cohort, our discovered signature of various miRNAs may behave as more specific and sensitive plasma biomarkers for SOC, EAOC as well as endometriosis cases.

### 8. REFERENCES

1. Siegel R, Naishadham D, Jemal A. Cancer statistics, 2012. CA: A Cancer Journal for Clinicians 2012; 62:10-29.
2. Clarke-Pearson DL. Screening for Ovarian Cancer. N Engl J Med 2009; 361:170-7.
3. Engel J, Eckel R, Schubert-Fritschle G, et al. Moderate progress for ovarian cancer in the last 20 years: prolongation of survival, but no improvement in the cure rate. European Journal of Cancer 2002; 38:2435-45.
4. Bast Jr R C, Badgwell D, Lu Z, et al. New tumor markers: CA125 and beyond. International Journal of Gynecological Cancer 2005; 15:274-81.
5. Gagnon A, Ye B. Discovery and application of protein biomarkers for ovarian cancer. Current Opinion in Obstetrics and Gynecology 2008; 20:9-13.
6. Valencia-Sanchez M A, Liu J, Hannon G J, Parker R. Control of translation and mRNA degradation by miRNAs and siRNAs. Genes & Development 2006; 20:515-24.
7. Ventura A, Jacks T. MicroRNAs and Cancer: Short RNAs Go a Long Way. Cell 2009; 136:586-91.
8. Iorio M V, Visone R, Di Leva G, et al. MicroRNA Signatures in Human. Ovarian Cancer. Cancer Res 2007; 67:8699-707.
9. Garzon R, Calin G A, Croce C M. MicroRNAs in Cancer. Annual Review of Medicine 2009; 60:167-79.
10. Lu J, Getz G, Miska E A, et al. MicroRNA expression profiles classify human cancers. Nature 2005; 435:834-8.
11. Gilad S, Meiri E, Yogev Y, et al. Serum MicroRNAs Are Promising Novel Biomarkers. PLoS ONE 2008; 3:e3148.
12. Mitchell P S, Parkin R K, Kroh E M, et al. Circulating microRNAs as stable blood-based markers for cancer detection. Proceedings of the National Academy of Sciences 2008; 105:10513-8.
13. Hu Z, Chen X, Zhao Y, et al. Serum MicroRNA Signatures Identified in a Genome-Wide Serum MicroRNA Expression Profiling Predict Survival of Non-Small-Cell Lung Cancer. J Clin Oncol 2010; 28:1721-6.
14. Wang J, Chen J, Chang P, et al. MicroRNAs in Plasma of Pancreatic Ductal Adenocarcinoma Patients as Novel Blood-Based Biomarkers of Disease. Cancer Prevention Research 2009; 2:807-13.
15. Pearce C L, Templeman C, Rossing M A, et al, Association between endometriosis and risk of histological subtypes of ovarian cancer: a pooled analysis of case-control studies. The Lancet Oncology 2012; 13:385-94.
16. Ness R B. Endometriosis and ovarian cancer: Thoughts on shared pathophysiology. American journal of obstetrics and gynecology 2003; 189:280-94.
17. Giudice L C. Endometriosis. N Engl J Med 2010; 362:2389-98.
18. Garry R. Endometriosis: an invasive disease. Gynaecological Endoscopy 2001; 10:79-82.
19. Oral E, Ilvan S, Tustas E, et al. Prevalence of endometriosis in malignant epithelial ovary tumours. European Journal of Obstetrics & Gynecology and Reproductive Biology 2003; 109:97-101.
20. Fukunaga M, Nomura K, Ishikawa E, Ushigome S. Ovarian atypical endometriosis: its close association with malignant epithelial tumours. Histopathology 1997; 30:249-55.
21. Varma R, Rollason T, Gupta J K, Maher E R. Endometriosis and the neoplastic process. Reproduction 2004; 127:293-304.
22. Nishida M, Watanabe K, Sato N, Ichikawa Y. Malignant transformation of ovarian endometriosis. Gynecol Obstet Invest 2000; 50:18-25.
23. Seidman J D. Prognostic importance of hyperplasia and atypia in endometriosis. Int J Gynecol Pathol 1996; 15:1-9.
24. Schmittgen T D, Livak K J. Analyzing real-time PCR data by the comparative C T method. Nat Protocols 2008; 3:1101-8.
25. Dinulescu D M, Ince T A, Quade B J, Shafer S A, Crowley D, Jacks T. Role of K-ras and Pten in the development of mouse models of endometriosis and endometrioid ovarian cancer. Nat Med 2005; 11:63-70.
26. Budiu R A, Diaconu I, Chrissluis R, Dricu A, Edwards R P, Vlad A M. A conditional mouse model for human MUC1-positive endometriosis shows the presence of anti-MUC1 antibodies and Foxp3+ regulatory T cells. Disease Models & Mechanisms 2009; 2:593-603.
27. Gail M H, Fears T R, Hoover R N, et al. Reproducibility studies and interlaboratory concordance for assays of serum hormone levels: estrone, estradiol, estrone sulfate, and progesterone. Cancer Epidemiology Biomarkers & Prevention 1996; 5:835-44.
28. Benjamini Y, Hochberg Y. Controlling the False Discovery Rate—a Practical and Powerful Approach to Multiple Testing. Journal of the Royal Statistical Society Series B-Methodological 1995; 57:289-300.
29. McLachlan G J. Cluster analysis and related techniques in medical research. Stat Methods Med Res 1992; 1:27-48.
30. McLachlan G J. Discriminant analysis and statistical pattern recognition. New York: Wiley; 1992.
31. Cannistra S A. Cancer of the Ovary. N Engl J Med 2004; 351:2519-29.
32. Dahiya N, Morin P J. MicroRNAs in ovarian carcinomas. Endocrine-Related Cancer 2010; 17:F77-F89.
33. Geiss G K, Bumgarner R E, Birditt B, et al. Direct multiplexed measurement of gene expression with color-coded probe pairs. Nat Biotech 2008; 26:317-25.
34. Wiegand K C, Shah S P, Al-Agha O M, et al. ARID1A Mutations in Endometriosis-Associated Ovarian Carcinomas. New England Journal of Medicine 2010; 363:1532-43.
35. Dzatic-Smiljkovic O, Vasiljevic M, Djukic M, Vugdelic R, Vugdelic J. Frequency of ovarian endometriosis in epithelial ovarian cancer patients. Clin Exp Obstet Gynecol 2011; 38:394-8.
36. Cramer D W, Bast R C, Berg C D, et al. Ovarian Cancer Biomarker Performance in Prostate, Lung, Colorectal, and Ovarian Cancer Screening Trial Specimens. Cancer Prevention Research 2011; 4:365-74.
37. Moss E L, Hollingworth J, Reynolds T M. The role of CA125 in clinical practice. J Clin Pathol 2005; 58:308-12.
38. Kobel M, Kalloger S E, Boyd N, et al. Ovarian Carcinoma Subtypes Are Different Diseases: Implications for Biomarker Studies. PLoS Med 2008; 5:e232.
39. Laschke M W, Menger M D. Anti-angiogenic treatment strategies for the therapy of endometriosis. Human Reproduction Update 2012; 18:682-702.
40. Kandalaft L E, Motz G T, Busch J, Coukos G. Angiogenesis and the tumor vasculature as antitumor immune modulators: the role of vascular endothelial growth factor and endothelin. Curr Top Microbiol Immunol 2011; 344:129-48.
41. Turchinovich A, Weiz L, Burwinkel B. Extracellular miRNAs: the mystery of their origin and function. Trends in biochemical sciences 2012; 37:460-5.
42. Nezhat F, Datta M S, Hanson V, Pejovic T, Nezhat C, Nezhat C. The relationship of endometriosis and ovarian malignancy: a review. Fertility and sterility 2008; 90:1559-70.
43. Wei J-J, William J, Bulun S. Endometriosis and Ovarian Cancer: A Review of Clinical, Pathologic, and Molecular Aspects. International Journal of Gynecologic Pathology 2011; 30:553-68.
44. Wyman S K, Parkin R K, Mitchell P S, et al. Repertoire of microRNAs in Epithelial Ovarian Cancer as Determined by Next Generation Sequencing of Small RNA cDNA Libraries. PLoS ONE 2009; 4:e5311.
45. Krichevsky A M, Gabriely G. miR-21: a small multi-faceted RNA. Journal of Cellular and Molecular Medicine 2009; 13:39-53.
46. Liu X-G, Zhu W-Y, Huang Y-Y, et al. High expression of serum miR-21 and tumor miR-200c associated with poor prognosis in patients with lung cancer. Medical Oncology 2012; 29:618-26.
47. Li B-s, Zhao Y-l, Guo G, et al. Plasma microRNAs, miR-223, miR-21 and miR-218, as Novel Potential Biomarkers for Gastric Cancer Detection. PLoS ONE 2012; 7:e41629.
48. Schrauder M G, Strick R, Schulz-Wendtland Rd, et al. Circulating Micro-RNAs as Potential Blood-Based Markers for Early Stage Breast Cancer Detection. PLoS ONE 2012; 7:e29770.
49. Resnick K E, Alder H, Hagan J P, Richardson D L, Croce C M, Cohn D E. The detection of differentially expressed microRNAs from the serum of ovarian cancer patients using a novel real-time PCR platform. Gynecologic Oncology 2009; 112:55-9.
50. Taylor D D, Gercel-Taylor C. MicroRNA signatures of tumor-derived exosomes as diagnostic biomarkers of ovarian cancer. Gynecologic Oncology 2008; 110:13-21.
51. Jemal, A., et al., Cancer statistics, 2008. CA Cancer J Clin, 2008.58(2): p. 71-96.
52. Kobayashi, H., et al., Ovarian endometrioma—risks factors of ovarian cancer development. Eur J Obstet Gynecol Reprod Biol, 2008.138(2): p. 187-93.
53. Kobayashi, H., et al., Risk of developing ovarian cancer among women with ovarian endometrioma: a cohort study in Shizuoka, Japan. Int J Gynecol Cancer, 2007. 17(1): p. 37-43.
54. Ogawa, S., et al., Ovarian endometriosis associated with ovarian carcinoma: a clinicopathological and immunohistochemical study. Gynecol Oncol, 2000. 77(2): p. 298-304.
55 Vigano, P., et al., Molecular mechanisms and biological plausibility underlying the malignant transformation of endometriosis: a critical analysis. Hum Reprod Update, 2006. 12(1): p. 77-89.
56. Nagle, C. M., et al., Endometrioid and clear cell ovarian cancers: a comparative analysis of risk factors. Eur J Cancer, 2008. 44(16): p. 2477-84.
57. Gourley, C., Link between endometriosis and ovarian-cancer subtypes. Lancet Oncol.
58. Sundaralingam, A., Endometriosis is associated with an increased risk of ovarian cancer, study shows. BMJ. 344: p. e1363.
59. Tadashi, T., No evidence of endometriosis within serous and mucinous tumors of the ovary. Int J Clin Exp Pathol. 5(2): p. 140-2.
60. Suh, K. S., et al., Ovarian cancer biomarkers for molecular biosensors and translational medicine. Expert Rev Mol Diagn. 10(8): p. 1069-83.
61. Samartzis, E. P., et al., Loss of ARID1A/BAF250a-expression in endometriosis: a biomarker for risk of carcinogenic transformation? Mod Pathol.
62. Jones, S., et al., Frequent mutations of chromatin remodeling gene ARID1A in ovarian clear cell carcinoma. Science. 330(6001): p. 228-31.
63. Cho, W. C., MicroRNAs: potential biomarkers for cancer diagnosis, prognosis and targets for therapy. Int J Biochem Cell Biol. 42(8): p. 1273-81.
64. Koturbash, I., et al., Small molecules with big effects: the role of the microRNAome in cancer and carcinogenesis. Mutat Res, 722(2): p. 94-105.
65. Teague, E. M., C. G. Print, and M. L. Hull, The role of microRNAs in endometriosis and associated reproductive conditions. Hum Reprod Update. 16(2): p. 142-65.
66. Arimoto, T., et al., Genome-wide cDNA microarray analysis of gene-expression profiles involved in ovarian endometriosis. Int J Oncol, 2003. 22(3): p. 551-60.
67, Eyster, K. M., et al., Whole genome deoxyribonucleic acid microarray analysis of gene expression in ectopic versus eutopic endometrium. Fertil Steril, 2007. 88(6): p. 1505-33.
68. Hever, A., et al., Human endometriosis is associated with plasma cells and overexpression of B lymphocyte stimulator. Proc Natl Acad Sci USA, 2007. 104(30): p. 12451-6.
69. Ohisson Teague, E M., et al., MicroRNA-regulated pathways associated with endometriosis. Mol Endocrinol, 2009.23(2): p. 265-75.
70. Borghese, B., et al., Gene expression profile for ectopic versus eutopic endometrium provides new insights into endometriosis oncogenic potential. Mol Endocrinol, 2008.22(11): p. 2557-62.
71. Hull, M. L., et al., Endometrial-peritoneal interactions during endometriotic lesion establishment. Am J Pathol, 2008.173(3): p. 700-15.
72. Ota, H., et al., Distribution of cyclooxygenase-2 in eutopic and ectopic endometrium in endometriosis and adenomyosis. Hum Reprod, 2001. 16(3): p. 561-6.
73. Braun, D. P., et al., Quantitative expression of apoptosis-regulating genes in endometrium from women with and without endometriosis. Fertil Steril, 2007. 87(2): p. 263-8.
74. Elyakim, E., et al., hsa-miR-191 is a candidate oncogene target for hepatocellular carcinoma therapy. Cancer Res. 70(20): p. 8077-87.
75. Xi, Y., et al., Prognostic Values of microRNAs in Colorectal Cancer. Biomark Insights, 2006.2: p. 113-121.
76. United States Patent Application No. US 2010/0249213.
77. Ach et al., BMC Biotechnology 2008, 8:69.
78. Chen et al., 2005, Nuc Acids Res 33:e179.
79. Liang et al., 2007, BMC Genomics 8:166.
80. Raymond et al., 2005, RNA 11:1737-1744.
81. Landgraf et al., 2007, Cell 129:1401-1414.
82. Mattie et al., 2006, Mol Cancer 5:24.
83. Bloomston et al., 2007, JAMA 297:1901-1908.
84. Porkka et al., 2007, Cancer Res 67:6130-6135.
85. Calin et al., 2004, Proc Natl Acad Sci USA 101:11755-11760.
86. Volinia et al., 2006, Proc Natl Acad Sci USA 103:2257-2261.
87. Wang et al., 2007, RNA 13:151-159.

Various publications are cited herein, the contents of each of which are hereby incorporated by reference in their entireties.

What is claimed is:

1. A kit for diagnosing endometriosis, endometriosis-associated ovarian cancer, or serous ovarian cancer, comprising measuring means for one or more of mir 15b, mir 16, mir 21, mir 191, mir 195, mir 362-5p, mir 376a, mir 628-3p, mir 744, mir 766, mir 1246, mir 1274a, mir 1308, mir 1915, mir 1973, mir 1974, mir 1975, mir 1977, mir 1978, mir 1979, mir 4284 where the measuring means is either (i) for each biomarker to be measured, a pair of oligonucleotide primers that may be used to amplify the biomarker or (ii) a microarray where said biomarker(s) constitute at least 10 percent of the species of biomarkers represented on the microarray.

2. The kit according to claim 1, for use in distinguishing between a healthy subject, a subject having endometriosis, a subject having endometriosis associated ovarian cancer and a subject having serous ovarian cancer, comprising measuring means for mir 16, mir 21 and mir 191 and at least one of mir 195, mir 4284, mir 362-5p, mir 1274a, mir 628-3p, mir 1915 and mir 1975 and combinations thereof.

3. The kit according to claim 2, further comprising a healthy control sample.

4. The kit according to claim 2, further comprising an endometriosis control sample.

5. The kit according to claim 2, further comprising an endometriosis associated ovarian control sample.

6. The kit according to claim 2, further comprising a serous ovarian cancer control sample.

7. The kit according to claim 1, for use in distinguishing between a healthy subject and a subject having endometriosis, comprising measuring means for one or more biomarker selected from the group consisting of mir 16; mir 15b; mir 191; mir 195; mir 1973; mir 1974; mir 1977, mir 1978, mir 1979, mir 4284 and mir 362-5p and combinations thereof.

8. The kit according to claim 7, further comprising a healthy control sample.

9. The kit according to claim 7, further comprising an endometriosis control sample.

10. The kit according to claim 1, for use in distinguishing between a healthy subject and a subject with endometriosis-associated ovarian cancer comprising measuring means for one or more biomarker selected from the group consisting of mir 16, mir 21, mir 15b, mir 191, mir 195, mir 1973, mir 1974, mir 1977, mir 1979, mir 766 and mir 4284 and combinations thereof.

11. The kit according to claim 10, further comprising a healthy control sample.

12. The kit according to claim 10, further comprising an endometriosis-associated ovarian cancer control sample.

13. The kit according to claim 1, for use in distinguishing between a healthy subject and a subject with serous ovarian cancer comprising measuring means for one or more biomarker selected from the group consisting of mir 4284, mir 1974, mir 16, mir 1977, mir 1975, mir 195, mir 1978, mir 21, mir 362-5p, mir 15b, mir 1308 and mir 191 and combinations thereof.

14. The kit according to claim 13, further comprising a healthy control sample.

15. The kit according to claim 13, further comprising an serous ovarian cancer control sample.

16. The kit according to claim 1, for use in distinguishing between a subject with endometriosis and a subject with endometriosis-associated ovarian cancer comprising measuring means for one or more biomarker selected from the group consisting of mir 362-5p, mir 1274a, mir-21, mir 766, mir 1975, mir 1308, mir 191, mir 744, mir 376a, and mir 1246 and combinations thereof.

17. The kit according to claim 16, further comprising an endometriosis control sample.

18. The kit according to claim 16, further comprising an endometriosis-associated ovarian cancer control sample.

19. The kit according to claim 1 for use in distinguishing between a subject with endometriosis and a subject with serous ovarian cancer comprising measuring means for one or more biomarker selected from the group consisting of mir-1915, mir 362-5p, and combinations thereof.

20. The kit according to claim 19, further comprising an endometriosis control sample.

21. The kit according to claim 19, further comprising a serous ovarian cancer control sample.

22. The kit according to claim 1 for distinguishing between endometriosis-associated ovarian cancer and serous ovarian cancer comprising measuring means for one or more biomarker selected from the group consisting of mir 21, mir 16, mir 191, mir 15b, mir 1975, mir 1246, mir 362-5p, mir 1979, mir 1973 and mir 195, and combinations thereof.

23. The kit according to claim 22, further comprising an endometriosis-associated ovarian cancer control sample.

24. The kit according to claim 22, further comprising a serous ovarian cancer.

25. A method of diagnosing a subject, comprising (a) obtaining a plasma sample from the subject; (b) purifying nucleic acid from the sample; (c) amplifying, from the nucleic acid, one or more microRNA biomarker that distinguishes between a healthy subject and (i) a subject having endometriosis; (ii) a subject with endometriosis associated ovarian cancer; and (iii) a subject having serous ovarian cancer; (d) measuring the level(s) of the one or more microRNA amplified according to step (c); (e) comparing the level of the one or more biomarker in the plasma sample to a control level or control levels; and (f) reporting if a diagnosis of endometriosis, endometriosis associated ovarian cancer or serous ovarian cancer is indicated.

26. A method of evaluating whether a subject having endometriosis has developed ovarian cancer, comprising (a) obtaining a plasma sample from the subject; (b) purifying nucleic acid from the sample; (c) amplifying, from the nucleic acid, one or more microRNA biomarker that distinguishes between a subject with endometriosis and (i) a subject with endometriosis associated ovarian cancer and/or (ii) a subject with serous ovarian cancer; (d) measuring the level(s) of the one or more microRNA amplified according to step (c); (e) comparing the level of the one or more biomarker in the plasma sample to a control level or control levels; and (f) reporting if a diagnosis of endometriosis associated ovarian cancer or serous ovarian cancer is indicated.
